SEQUENCE LISTING (98 sequences)

SEQ ID NO: 1 - DNA, 20 bp, synthetic construct
  source 1..20: mol_type = other DNA
agagtgagtagtagtggagt

SEQ ID NO: 2 - DNA, 20 bp, synthetic construct
  source 1..20: mol_type = other DNA
tgtgatggaagttagagggt

SEQ ID NO: 3 - DNA, 20 bp, synthetic construct
  source 1..20: mol_type = other DNA
tgaaaggaatgggttgtggt

SEQ ID NO: 4 - DNA, 20 bp, synthetic construct
  source 1..20: mol_type = other DNA
gggttgattagtggtagaaa

SEQ ID NO: 5 - DNA, 20 bp, synthetic construct
  source 1..20: mol_type = other DNA
tgtggagggattgaaggata

SEQ ID NO: 6 - DNA, 20 bp, synthetic construct
  source 1..20: mol_type = other DNA
gggagaatgaggtgtaatgt

SEQ ID NO: 7 - DNA, 20 bp, synthetic construct
  source 1..20: mol_type = other DNA
tagagttgatagagggagaa

SEQ ID NO: 8 - DNA, 20 bp, synthetic construct
  source 1..20: mol_type = other DNA
gatgatgtagtagtaagggt

SEQ ID NO: 9 - DNA, 20 bp, synthetic construct
  source 1..20: mol_type = other DNA
ggagtagttggttgttagga

SEQ ID NO: 10 - DNA, 20 bp, synthetic construct
  source 1..20: mol_type = other DNA
aggaggagggtaatgataga

SEQ ID NO: 11 - DNA, 20 bp, synthetic construct
  source 1..20: mol_type = other DNA
gagggtttgtaaggtgaata

SEQ ID NO: 12 - DNA, 20 bp, synthetic construct
  source 1..20: mol_type = other DNA
gaagtgaggtgattgagtga

SEQ ID NO: 13 - DNA, 20 bp, synthetic construct
  source 1..20: mol_type = other DNA
ggtattatgtaggaaggtgg

SEQ ID NO: 14 - DNA, 20 bp, synthetic construct
  source 1..20: mol_type = other DNA
gagaagtggttgtagagtgt

SEQ ID NO: 15 - DNA, 20 bp, synthetic construct
  source 1..20: mol_type = other DNA
ggttagtaggttgtggtgtt

SEQ ID NO: 16 - DNA, 20 bp, synthetic construct
  source 1..20: mol_type = other DNA
gtataaggtgattggtggtg

SEQ ID NO: 17 - DNA, 20 bp, synthetic construct
  source 1..20: mol_type = other DNA
ggagtaggttgatgtgtagt

SEQ ID NO: 18 - DNA, 20 bp, synthetic construct
  source 1..20: mol_type = other DNA
gagtgtgtgttaaggtaggt

SEQ ID NO: 19 - DNA, 20 bp, synthetic construct
  source 1..20: mol_type = other DNA
tggttagaggttagtggttg

SEQ ID NO: 20 - DNA, 20 bp, synthetic construct
  source 1..20: mol_type = other DNA
atgtgagtggtgagaatgtg

SEQ ID NO: 21 - DNA, 20 bp, synthetic construct
  source 1..20: mol_type = other DNA
ggtggttgattaaggatggt

SEQ ID NO: 22 - DNA, 20 bp, synthetic construct
  source 1..20: mol_type = other DNA
ggaggttagaatttgtgagg

SEQ ID NO: 23 - DNA, 20 bp, synthetic construct
  source 1..20: mol_type = other DNA
gagagaggattaggtattgg

SEQ ID NO: 24 - DNA, 20 bp, synthetic construct
  source 1..20: mol_type = other DNA
gtaggtgttatgttaggagg

SEQ ID NO: 25 - DNA, 133 bp, synthetic construct
  source 1..133: mol_type = other DNA
tggccgtcgattccgtgaatagagtgagtagtagtggagtaagagtgagtagtagtggagtaggtcttcatggtgcatgtggttcatcaccaaagagtgagtagtagtggagtgcagaatttcctggtgcggg

SEQ ID NO: 26 - DNA, 133 bp, synthetic construct
  source 1..133: mol_type = other DNA
tggccgtcgattccgtgaattgtgatggaagttagagggtatgtgatggaagttagagggtagaggcttttctaggaccttttcttctggatatgtgatggaagttagagggtgcagaatttcctggtgcggg

SEQ ID NO: 27 - DNA, 133 bp, synthetic construct
  source 1..133: mol_type = other DNA
tggccgtcgattccgtgaattgaaaggaatgggttgtggtaaacagatctcactgcacacgggcatatctcatgaaaggaatgggttgtggtatgaaaggaatgggttgtggtgcagaatttcctggtgcggg

SEQ ID NO: 28 - DNA, 133 bp, synthetic construct
  source 1..133: mol_type = other DNA
tggccgtcgattccgtgaatgggttgattagtggtagaaaatggaaggagagagagattctgtccgcggccagggttgattagtggtagaaaagggttgattagtggtagaaagcagaatttcctggtgcggg

SEQ ID NO: 29 - DNA, 133 bp, synthetic construct
  source 1..133: mol_type = other DNA
tggccgtcgattccgtgaattgtggagggattgaaggataatgtggagggattgaaggataaggaggtggaatctttttttgtacgaggtcaatgtggagggattgaaggatagcagaatttcctggtgcggg

SEQ ID NO: 30 - DNA, 133 bp, synthetic construct
  source 1..133: mol_type = other DNA
tggccgtcgattccgtgaatgggagaatgaggtgtaatgtagggagaatgaggtgtaatgtaccctgtctcgaaaagcaaacaaataacagcagggagaatgaggtgtaatgtgcagaatttcctggtgcggg

SEQ ID NO: 31 - DNA, 133 bp, synthetic construct
  source 1..133: mol_type = other DNA
tggccgtcgattccgtgaattagagttgatagagggagaaaatgccactaccacggaaacctgaggtttttatagagttgatagagggagaaatagagttgatagagggagaagcagaatttcctggtgcggg

SEQ ID NO: 32 - DNA, 133 bp, synthetic construct
  source 1..133: mol_type = other DNA
tggccgtcgattccgtgaatgatgatgtagtagtaagggtacttcaccagacagccttctataaagctgaaagatgatgtagtagtaagggtagatgatgtagtagtaagggtgcagaatttcctggtgcggg

SEQ ID NO: 33 - DNA, 133 bp, synthetic construct
  source 1..133: mol_type = other DNA
tggccgtcgattccgtgaatggagtagttggttgttaggaaggagtagttggttgttaggaaggtatagatgggccccatttcctcttactcaggagtagttggttgttaggagcagaatttcctggtgcggg

SEQ ID NO: 34 - DNA, 133 bp, synthetic construct
  source 1..133: mol_type = other DNA
tggccgtcgattccgtgaataggaggagggtaatgatagaatcttgatgctgctggagtctcatcagctgaaaggaggagggtaatgatagaaaggaggagggtaatgatagagcagaatttcctggtgcggg

SEQ ID NO: 35 - DNA, 133 bp, synthetic construct
  source 1..133: mol_type = other DNA
tggccgtcgattccgtgaatgagggtttgtaaggtgaataagagggtttgtaaggtgaataacgttgtatgtgcaggtttgtgggaacaaatagagggtttgtaaggtgaatagcagaatttcctggtgcggg

SEQ ID NO: 36 - DNA, 133 bp, synthetic construct
  source 1..133: mol_type = other DNA
tggccgtcgattccgtgaatgaagtgaggtgattgagtgaaaggatgaggatggtggtgaaggccttggttagaagtgaggtgattgagtgaagaagtgaggtgattgagtgagcagaatttcctggtgcggg

SEQ ID NO: 37 - DNA, 133 bp, synthetic construct
  source 1..133: mol_type = other DNA
tggccgtcgattccgtgaatggtattatgtaggaaggtggagagactagcctgtgaccaatttaaccacagaggtattatgtaggaaggtggaggtattatgtaggaaggtgggcagaatttcctggtgcggg

SEQ ID NO: 38 - DNA, 133 bp, synthetic construct
  source 1..133: mol_type = other DNA
tggccgtcgattccgtgaatgagaagtggttgtagagtgtaggcggttcattcgagccatgttaatttagtagagaagtggttgtagagtgtagagaagtggttgtagagtgtgcagaatttcctggtgcggg

SEQ ID NO: 39 - DNA, 133 bp, synthetic construct
  source 1..133: mol_type = other DNA
tggccgtcgattccgtgaatggttagtaggttgtggtgttaatccctcacagctccacaaaactgcgggccaggttagtaggttgtggtgttaggttagtaggttgtggtgttgcagaatttcctggtgcggg

SEQ ID NO: 40 - DNA, 133 bp, synthetic construct
  source 1..133: mol_type = other DNA
tggccgtcgattccgtgaatgtataaggtgattggtggtgagagccagcaaaccggcagaaaagctgctgtagtataaggtgattggtggtgagtataaggtgattggtggtggcagaatttcctggtgcggg

SEQ ID NO: 41 - DNA, 133 bp, synthetic construct
  source 1..133: mol_type = other DNA
tggccgtcgattccgtgaatggagtaggttgatgtgtagtaggagtaggttgatgtgtagtaggtttgctcattctgttgagcgcgatacataggagtaggttgatgtgtagtgcagaatttcctggtgcggg

SEQ ID NO: 42 - DNA, 133 bp, synthetic construct
  source 1..133: mol_type = other DNA
tggccgtcgattccgtgaatgagtgtgtgttaaggtaggtagagtgtgtgttaaggtaggtaatgcaaagacattggccagcattgctgtgaagagtgtgtgttaaggtaggtgcagaatttcctggtgcggg

SEQ ID NO: 43 - DNA, 133 bp, synthetic construct
  source 1..133: mol_type = other DNA
tggccgtcgattccgtgaattggttagaggttagtggttgacccacccccaataatacaaataaaacccctatggttagaggttagtggttgatggttagaggttagtggttggcagaatttcctggtgcggg

SEQ ID NO: 44 - DNA, 133 bp, synthetic construct
  source 1..133: mol_type = other DNA
tggccgtcgattccgtgaatatgtgagtggtgagaatgtgagttctgctgtgccaaccgcttgcgttcgtcaatgtgagtggtgagaatgtgaatgtgagtggtgagaatgtggcagaatttcctggtgcggg

SEQ ID NO: 45 - DNA, 133 bp, synthetic construct
  source 1..133: mol_type = other DNA
tggccgtcgattccgtgaatggtggttgattaaggatggtacgggagaacacggtgggagatgatctcatgaggtggttgattaaggatggtaggtggttgattaaggatggtgcagaatttcctggtgcggg

SEQ ID NO: 46 - DNA, 133 bp, synthetic construct
  source 1..133: mol_type = other DNA
tggccgtcgattccgtgaatggaggttagaatttgtgaggaatgttagttgggacagcagcatccacagcaaggaggttagaatttgtgaggaggaggttagaatttgtgagggcagaatttcctggtgcggg

SEQ ID NO: 47 - DNA, 133 bp, synthetic construct
  source 1..133: mol_type = other DNA
tggccgtcgattccgtgaatgagagaggattaggtattggagagagaggattaggtattggattggtaggaggcagtggcttgtcgtcctccagagagaggattaggtattgggcagaatttcctggtgcggg

SEQ ID NO: 48 - DNA, 133 bp, synthetic construct
  source 1..133: mol_type = other DNA
tggccgtcgattccgtgaatgtaggtgttatgttaggaggaccaagtgcggttttaccaaggttactattgagtaggtgttatgttaggaggagtaggtgttatgttaggagggcagaatttcctggtgcggg

SEQ ID NO: 49 - DNA, 20 bp, synthetic construct
  source 1..20: mol_type = other DNA
tggccgtcgattccgtgaat

SEQ ID NO: 50 - DNA, 20 bp, synthetic construct
  source 1..20: mol_type = other DNA
gcagaatttcctggtgcggg

SEQ ID NO: 51 - DNA, 29 bp, synthetic construct
  source 1..29: mol_type = other DNA
ggtcttcatggtgcatgtggttcatcacc

SEQ ID NO: 52 - DNA, 29 bp, synthetic construct
  source 1..29: mol_type = other DNA
gaggcttttctaggaccttttcttctgga

SEQ ID NO: 53 - DNA, 29 bp, synthetic construct
  source 1..29: mol_type = other DNA
aacagatctcactgcacacgggcatatct

SEQ ID NO: 54 - DNA, 29 bp, synthetic construct
  source 1..29: mol_type = other DNA
tggaaggagagagagattctgtccgcggc

SEQ ID NO: 55 - DNA, 29 bp, synthetic construct
  source 1..29: mol_type = other DNA
ggaggtggaatctttttttgtacgaggtc

SEQ ID NO: 56 - DNA, 29 bp, synthetic construct
  source 1..29: mol_type = other DNA
ccctgtctcgaaaagcaaacaaataacag

SEQ ID NO: 57 - DNA, 29 bp, synthetic construct
  source 1..29: mol_type = other DNA
atgccactaccacggaaacctgaggtttt

SEQ ID NO: 58 - DNA, 29 bp, synthetic construct
  source 1..29: mol_type = other DNA
cttcaccagacagccttctataaagctga

SEQ ID NO: 59 - DNA, 29 bp, synthetic construct
  source 1..29: mol_type = other DNA
ggtatagatgggccccatttcctcttact

SEQ ID NO: 60 - DNA, 29 bp, synthetic construct
  source 1..29: mol_type = other DNA
tcttgatgctgctggagtctcatcagctg

SEQ ID NO: 61 - DNA, 29 bp, synthetic construct
  source 1..29: mol_type = other DNA
cgttgtatgtgcaggtttgtgggaacaaa

SEQ ID NO: 62 - DNA, 29 bp, synthetic construct
  source 1..29: mol_type = other DNA
aggatgaggatggtggtgaaggccttggt

SEQ ID NO: 63 - DNA, 29 bp, synthetic construct
  source 1..29: mol_type = other DNA
gagactagcctgtgaccaatttaaccaca

SEQ ID NO: 64 - DNA, 29 bp, synthetic construct
  source 1..29: mol_type = other DNA
ggcggttcattcgagccatgttaatttag

SEQ ID NO: 65 - DNA, 29 bp, synthetic construct
  source 1..29: mol_type = other DNA
atccctcacagctccacaaaactgcgggc

SEQ ID NO: 66 - DNA, 29 bp, synthetic construct
  source 1..29: mol_type = other DNA
gagccagcaaaccggcagaaaagctgctg

SEQ ID NO: 67 - DNA, 29 bp, synthetic construct
  source 1..29: mol_type = other DNA
ggtttgctcattctgttgagcgcgataca

SEQ ID NO: 68 - DNA, 29 bp, synthetic construct
  source 1..29: mol_type = other DNA
atgcaaagacattggccagcattgctgtg

SEQ ID NO: 69 - DNA, 29 bp, synthetic construct
  source 1..29: mol_type = other DNA
cccacccccaataatacaaataaaacccc

SEQ ID NO: 70 - DNA, 29 bp, synthetic construct
  source 1..29: mol_type = other DNA
gttctgctgtgccaaccgcttgcgttcgt

SEQ ID NO: 71 - DNA, 29 bp, synthetic construct
  source 1..29: mol_type = other DNA
cgggagaacacggtgggagatgatctcat

SEQ ID NO: 72 - DNA, 29 bp, synthetic construct
  source 1..29: mol_type = other DNA
atgttagttgggacagcagcatccacagc

SEQ ID NO: 73 - DNA, 29 bp, synthetic construct
  source 1..29: mol_type = other DNA
ttggtaggaggcagtggcttgtcgtcctc

SEQ ID NO: 74 - DNA, 29 bp, synthetic construct
  source 1..29: mol_type = other DNA
ccaagtgcggttttaccaaggttactatt

SEQ ID NO: 75 - DNA, 20 bp, synthetic construct
  source 1..20: mol_type = other DNA
actccactactactcactct

SEQ ID NO: 76 - DNA, 20 bp, synthetic construct
  source 1..20: mol_type = other DNA
accctctaacttccatcaca

SEQ ID NO: 77 - DNA, 20 bp, synthetic construct
  source 1..20: mol_type = other DNA
accacaacccattcctttca

SEQ ID NO: 78 - DNA, 20 bp, synthetic construct
  source 1..20: mol_type = other DNA
tttctaccactaatcaaccc

SEQ ID NO: 79 - DNA, 20 bp, synthetic construct
  source 1..20: mol_type = other DNA
tatccttcaatccctccaca

SEQ ID NO: 80 - DNA, 20 bp, synthetic construct
  source 1..20: mol_type = other DNA
acattacacctcattctccc

SEQ ID NO: 81 - DNA, 20 bp, synthetic construct
  source 1..20: mol_type = other DNA
caaccactaacctctaacca

SEQ ID NO: 82 - DNA, 20 bp, synthetic construct
  source 1..20: mol_type = other DNA
cacattctcaccactcacat

SEQ ID NO: 83 - DNA, 20 bp, synthetic construct
  source 1..20: mol_type = other DNA
accatccttaatcaaccacc

SEQ ID NO: 84 - DNA, 20 bp, synthetic construct
  source 1..20: mol_type = other DNA
ttctccctctatcaactcta

SEQ ID NO: 85 - DNA, 20 bp, synthetic construct
  source 1..20: mol_type = other DNA
acccttactactacatcatc

SEQ ID NO: 86 - DNA, 20 bp, synthetic construct
  source 1..20: mol_type = other DNA
tcctaacaaccaactactcc

SEQ ID NO: 87 - DNA, 20 bp, synthetic construct
  source 1..20: mol_type = other DNA
tctatcattaccctcctcct

SEQ ID NO: 88 - DNA, 20 bp, synthetic construct
  source 1..20: mol_type = other DNA
tattcaccttacaaaccctc

SEQ ID NO: 89 - DNA, 20 bp, synthetic construct
  source 1..20: mol_type = other DNA
tcactcaatcacctcacttc

SEQ ID NO: 90 - DNA, 20 bp, synthetic construct
  source 1..20: mol_type = other DNA
cctcacaaattctaacctcc

SEQ ID NO: 91 - DNA, 20 bp, synthetic construct
  source 1..20: mol_type = other DNA
ccaatacctaatcctctctc

SEQ ID NO: 92 - DNA, 20 bp, synthetic construct
  source 1..20: mol_type = other DNA
cctcctaacataacacctac

SEQ ID NO: 93 - DNA, 20 bp, synthetic construct
  source 1..20: mol_type = other DNA
ccaccttcctacataatacc

SEQ ID NO: 94 - DNA, 20 bp, synthetic construct
  source 1..20: mol_type = other DNA
acactctacaaccacttctc

SEQ ID NO: 95 - DNA, 20 bp, synthetic construct
  source 1..20: mol_type = other DNA
aacaccacaacctactaacc

SEQ ID NO: 96 - DNA, 20 bp, synthetic construct
  source 1..20: mol_type = other DNA
caccaccaatcaccttatac

SEQ ID NO: 97 - DNA, 20 bp, synthetic construct
  source 1..20: mol_type = other DNA
actacacatcaacctactcc

SEQ ID NO: 98 - DNA, 20 bp, synthetic construct
  source 1..20: mol_type = other DNA
acctaccttaacacacactc

(19) United States
(12) Patent Application Publication
(10) Pub. No.: US 2024/0321393 A1
(43) Pub. Date: Sep. 26, 2024

(54) CELL-TYPE OPTIMIZATION METHOD AND SCANNER

(71) Applicant: The Regents of the University of California, Oakland, CA (US)

(72) Inventors: Roy WOLLMAN, Los Angeles, CA (US); Zachary HEMMINGER, Los Angeles, CA (US)

(73) Assignee: The Regents of the University of California, Oakland, CA (US)

(21) Appl. No.: 18/580,053

(22) PCT Filed: Jul. 27, 2022
(86) PCT No.: PCT/US22/74201

§ 371 Date: Jan. 17, 2024

Related U.S. Application Data
(60) Provisional application No. 63/226,660, filed on Jul. 28, 2021.

(51) Int. Cl. G16B 25/10; C12Q 1/6825; G01N 1/30; G01N 21/64; G01N 33/53; G01N 33/58; G01N 33/68; G16H 40/20

(52) U.S. Cl. CPC G16B 25/10; C12Q 1/6825; G01N 1/30; G01N 21/6456; G01N 33/53; G01N 33/58; G01N 33/6803; G16H 40/20

(57) ABSTRACT

Methods are described for cell-type mapping utilizing direct measurement of low-dimensional representation of single-cell transcriptomics with a supervised machine learning algorithm to spatially map cell types, bypassing the need to measure expression of single genes. Such methods are useful to identify locations of cell types in biological specimens for purposes including tissue based diagnostics.

### CROSS-REFERENCE TO RELATED APPLICATIONS

This application claims priority to U.S. provisional patent application Ser. No. 63/226,660, filed Jul. 28, 2021, the entire contents of which is incorporated herein by reference.

### FIELD

The present disclosure relates to methods of creating biological investigative protocols, and laboratory apparatus which implement such methods.

### INCORPORATION BY REFERENCE

The following documents and all others cited herein are incorporated by reference in their entireties: U.S. Pat. Nos. 4,946,778; 10,068,053; 10,706,955; U.S. Pub. No. 2020/0320355 A1; U.S. Pub. No. 2020/0152289 A1; WO 2017/075292 A1; Bracewell et al., Int'l Conf. Eng. Des., 24-27 Aug. 2009, 6-223 to 6-243; Caicedo et al., Nat. Methods, September 2017 14(9), 849-63; Guan et al., PLoS One, 2013 Dec. 20; 8(12):e83291; Yuste et al., Nat. Neurosci., December 2020, 23 1456-68.

### BACKGROUND

Beginning with Anton van Leeuwenhoek's microscopy observations in the mid-17th century, natural philosophers such as Robert Hooke noted that plant tissues seemed to “consist of a great many little Boxes” of which there were “in a Cubic Inch, above twelve hundred Millions . . . a thing almost incredible.” (R. Hooke, Micrographia (1655)). In the 19th century, Matthias Jakob Schleiden, Theodor Schwann, and later Rudolf Virchow, concluded that not only plants but also animals consist of cells having varying structure and function, and they developed a fundamental cell theory of biology.

Around the late 19th century, Camillo Golgi and Santiago Ramón y Cajal contributed to the systemization of cell biology by carefully staining, drawing, and cataloguing neurological cells, and naming the various cell types distinguished by their diverse visible morphologies.

Since these early findings, investigators have described countless different cell types. Modern cell biology has arrived at granular distinctions between cells, and numerous ways to describe and classify cell types, including, e.g., (a) anatomical morphology; (b) apparent physiological function; (c) protein markers; (d) genetic markers; (e) developmental origins and taxonomy; (f) epigenetic states and markers; (g) electrophysiology; (h) cross-species homology; and (i) combinations of any of these factors.

Lately, it has become well known that some cell types having identical morphology may sometimes be distinguished by molecular markers, physiological function, or both. (See, e.g., Hattar et al., SCIENCE, 8 Feb. 2002, 295(5557), 1065-70.) Consequently, classification frameworks have moved toward quantifiable molecular methods that can detect and/or visually label cells by their distinguishing molecular features.

While labeling a protein or a nucleic acid in a sample has become routine laboratory technique, it remains challenging to label large quantities of different protein and/or nucleic acid markers in complex tissues, such as brain neocortical tissue, such that all cell types may be readily resolved, identified, and distinguished. Investigators must carefully select individual or a limited set of molecular markers and labels, e.g., fluorophores, such that each cell type is clearly and distinguishably labeled. However, use of individual or limited sets of such markers or labels for mapping purposes cannot provide the power to distinguish among a plethora (e.g., hundreds to thousands) of cell types.

Spatial mapping of the three-dimensional organization of cells in complex tissues will be transformative towards understanding of biological organization of function. Recent advances in resolution, sensitivity, and throughput have made “spatial transcriptomics” the main tool for special cell-type mapping in study of the brain. However, the existing major spatial transcriptomics approaches are limited to thin two-dimensional sections.

Accordingly, there is a great need for reliable, efficient methods of optimizing cell labeling protocols; methods of analyzing and consolidating vast amounts of cell labeling information into topographically meaningful presentations, and apparatus capable of identifying and recording each cell's type and spatial position in the sample, all which could enable three-dimensional volumetric mapping. Such methods and apparatus would help develop universal, optimal labeling protocols using the best and fewest fluorophores labels possible, which will improve the efficacy, quality, and reproducibility of investigative research, and greatly aid in research of complex tissues. These research capability improvements will lead to new biological discoveries and medical treatments.

### SUMMARY

The present disclosure relates to methods of optimizing biological investigative protocols, and laboratory apparatus. More specifically, the present disclosure comprises a computational method for determining how to optimally label cells in a biological sample such that each cell type is distinguishable from all other cell types in the biological sample. The present method would ultimately permit investigators to reliably distinguish a large variety of differing cell types in a tissue sample using a minimal array of visual labels.

The present disclosure further comprises a scanner apparatus which can determine and record the position and labels on each cell in a biological sample, and determine or estimate the cell type at that location.

In an embodiment, the present disclosure comprises a method for identifying the specific locations of a plurality of specific cell types within a population of cells in a biological specimen, the method comprising the steps of:
(a) selecting the plurality of specific cell types within the specimen, based on the origin of the specimen and the known cell types anticipated to be present therein;
(b) determining among those specific cell types the known extent of expression or lack thereof of one or more known molecular markers of each specific cell type therein;
(c) establishing a relationship using a subset of the one or more molecular markers among all specific cell types therein, wherein a weight given to the extent of expression or lack thereof of each molecular marker (e.g., nucleic acid polymer(s) and/or protein marker(s)) by each of the specific cell types using a linear dimensionality reduction process differentiates each specific cell type from each other specific cell type;
(d) preparing a set of bivalent binding reagents and a set of labeled binding reagents, wherein each bivalent binding reagent comprises a molecular marker-binding region and at least one labeled-binding-reagent binding region, wherein one or more of each molecular marker-specific bivalent binding reagent is provided to bind to a specific molecular marker to an extent to differentiate the specific molecular marker from each other molecular marker, and wherein each labeled binding reagent is detectably labeled by individually and/or simultaneously detectable labels, such that the set of bivalent binding reagents and labeled binding reagents bound thereto, when bound to the subset of molecular markers expressed by each specific cell type in the sample, provides an extent of labeling that differentiates each specific cell type from each other specific cell type;
(e) incubating the specimen sequentially or simultaneously with the bivalent binding reagents and the labeled binding reagents;
(f) imaging positions throughout the specimen to detect the labeled binding reagents and extent of labeling thereof sequentially or simultaneously, to provide the positions of the subset of molecular markers and extent of expression thereof;
(g) correlating the extent of expression of the subset of molecular markers at positions throughout the specimen using the linear dimensionality reduction process based on the established relationship between the extent of expression of molecular markers or lack thereof by each of the specific cell types that differentiates each specific cell type from each other specific cell type, to establish a highest-probability estimate of the presence of a specific cell type at a specific location within the specimen; and
(h) identifying the specific locations of the specific estimated cell types within the specimen.

In one embodiment, the molecular markers are nucleic acid polymers. In a preferred embodiment, the nucleic acid polymers are RNA. In an alternative embodiment, the molecular markers are peptides, whole proteins, and/or protein fragments, which may comprise any of a peptide, nuclear protein, cytosolic protein, mitochondrial protein, secreted protein, cell-surface protein, receptor, transcription factor, antibody, or any combination thereof. In other embodiments, the molecular markers are any biological components that can be tagged in accordance with the disclosure herein. Non-limiting examples include metabolites, lipids, carbohydrates including polysaccharides, glycolipids, vitamins, fatty acids, co-factors, pigments, metals, or any other biochemicals or compounds, organic or inorganic, found within a biological system. The methods described herein for identifying exemplary molecules are equally applicable to markers that are not nucleic acids or proteins. Moreover, the methods may be used to concurrently identify more than one type of molecular marker in a specimen.

In one embodiment, steps (a), (b) and (c) are performed for a particular type of biological specimen wherein the specimen comprises a plurality of known cell types (e.g., known from the literature) and among the known cell types within the specimen from which the plurality are selected for locating, in step (a), the known molecular markers of each cell type is obtained from the literature, for step (b). Based upon the selection of molecular markers in step (b), step (c) may be carried out to identify the markers to be detected, and step (d) the design of the reagents. Thus, steps (a)-(d) are carried out for each particular type of specimen and cells therein of interest in locating. In one embodiment, the remainder of the steps are carried out on the specimen and analyzing the data collected from imaging the specimen.

In one embodiment, steps (a), (b) and (c) are carried out in silico.

In another embodiment, step (b) may further comprise organizing the specific cell types into a hierarchical taxonomy according to the plurality of known molecular markers. In some embodiments, the number of different detectable labels of step (d) may equal to the number of hierarchical levels of the hierarchical taxonomy.

In yet another embodiment, the dimensionality reduction process of step (c) is accomplished using principal component analysis. In still another embodiment, the dimensionality reduction process of step (c) is accomplished using recursive partitioning. In another embodiment, the dimensionality reduction process of step (c) is accomplished using artificial neural network to design the encoding. In an alternative embodiment, the dimensionality reduction process of step (c) is accomplished using discriminant projection non-negative matrix factorization (dPNMF). In an aspect, dPNMF comprises the steps of (i) fitting a dPNMF model to training data; (ii) fitting a classifier to one class per cell type; and (iii) creating a staining profile for each cell type according to a weighting, whereby the number of cell labels per molecular marker approximates the weighting. In an aspect, the classifier of step (ii) is a Naïve Bayesian classifier. In another aspect, artificial neural network classifiers are used. In another aspect, the K-nearest neighbors algorithm (KNN) is used.

In one embodiment, step (d) is accomplished using direction from step (c) as to the preparation of the set of bivalent binding reagents and labeled binding reagents. In one embodiment, the bivalent binding reagent is an oligonucleotide comprising a molecular marker-binding region and a labeled-binding-reagent-binding region. In one embodiment, multiple bivalent binding reagents are provided that bind to the same molecular marker. In some embodiment, the multiple bivalent reagents that bind to the same molecular marker have the same labeled-binding-reagent-binding region. In one embodiment the labeled binding reagent comprises a label and a region that binds to a bivalent binding reagent. In one embodiment, the labels of the labeled binding reagents are dyes that are individually detectable in a single location within the specimen using hyperspectral imaging. In some embodiments a labeled binding reagent has a single dye molecule bound thereto. In some embodiments a labeled binding reagent has a plurality of dye molecules bound thereto. In some embodiments, the plurality of dye molecules are the same dye or different dyes.

In some embodiment, oligonucleotide-based bivalent binding reagents comprise at least one molecular-marker binding region nucleic acid sequence, and at least one labeled-binding-reagent binding region nucleic acid sequence. In some embodiments a bivalent binding reagent may comprise two labeled-binding-reagent binding sequences, binding the same or different labeled binding reagents. In some embodiments a bivalent binding reagent may comprise three labeled-binding-reagent binding sequences. In some embodiments a bivalent binding reagent may comprise more than three labeled-binding-reagent binding sequences. Such plurality of labeled-binding-reagent binding sequences on a bivalent binding reagent may bind one or more of the same, or two different, or two of the same and one different, or three all different labeled binding reagents, or any other combination thereof. Such number of labeled-binding-reagent binding sequences will be provided in the calculations from step a, b and/or c as described herein.

In some embodiments, for purposes of amplifying the bivalent binding sequences, amplification sequences may be include in the bivalent binding reagents, which may be retained or removed before use in the staining methods disclosed herein. In some embodiments, a forward and reverse amplification nucleic acid sequence are included in the bivalent binding reagent. In some embodiment, the forward amplification sequence is provided at the 5′ end of the bivalent binding reagent, and the reverse amplification sequence at the 3′end.

In some embodiments, the bivalent binding sequence may comprise, from 5′ to 3′, a forward amplification sequence, a molecular-marker binding sequence, a labeled-binding-reagent binding sequence, and a reverse amplification sequence. In some embodiments the aforementioned sequence is cleaved to a, from 5′ to 3′, a molecular-marker binding sequence and a labeled-binding-reagent binding sequence.

In some embodiments, the bivalent binding sequence may comprise, from 5′ to 3′, a forward amplification sequence, a labeled-binding-reagent binding sequence, a molecular-marker binding sequence, and a reverse amplification sequence. In some embodiments the aforementioned sequence is cleaved to a, from 5′ to 3′, a labeled-binding-reagent binding sequence and a molecular-marker binding sequence.

In some embodiments, the bivalent binding sequence may comprise, from 5′ to 3′, a forward amplification sequence, a labeled-binding-reagent binding sequence, a labeled-binding-reagent binding sequence, a molecular-marker binding sequence, and a reverse amplification sequence. In some embodiments the aforementioned sequence is cleaved to a, from 5′ to 3′, a labeled-binding-reagent binding sequence, a labeled-binding-reagent-binding sequence, and a molecular-marker binding sequence.

In some embodiments, the bivalent binding sequence may comprise, from 5′ to 3′, a forward amplification sequence, a labeled-binding-reagent binding sequence, a labeled-binding-reagent binding sequence, a molecular-marker binding sequence, a labeled-binding-reagent binding region, and a reverse amplification sequence. In some embodiments the aforementioned sequence is cleaved to a, from 5′ to 3′, a labeled-binding-reagent binding sequence, a labeled-binding-reagent-binding sequence, a molecular-marker binding sequence, and a labeled-binding-reagent binding sequence.

In some embodiments, the bivalent binding sequence may comprise, from 5′ to 3′, a forward amplification sequence, a labeled-binding-reagent binding sequence, a molecular-marker binding sequence, a labeled-binding-reagent binding region, a labeled-binding-reagent binding sequence, and a reverse amplification sequence. In some embodiments the aforementioned sequence is cleaved to a, from 5′ to 3′, a labeled-binding-reagent binding sequence, a molecular-marker binding sequence, a labeled-binding-reagent-binding sequence, and a labeled-binding-reagent binding sequence.

In any of the bivalent binding reagents disclosed herein, additional nucleotides (e.g., A, C, G, and/or T) may be provided as spacers between the aforementioned regions, such as one or more A.

In some embodiments, bivalent binding reagents are provided that bind to different sequences on the same molecular marker; such multiple bivalent binding reagents for the same marker provide the weights that each molecular marker target contributes towards the total basis measurement.

In some embodiments, oligonucleotide-based labeled binding reagents comprise an oligonucleotide sequence that binds to a labeled-binding-reagent binding sequence of a bivalent binding reagent, and a detectable label such as a fluorescent dye. In some embodiments the dye is covalent bound to the oligonucleotide region of the labeled binding reagent. In some embodiments, the dye is reversibly linked to the oligonucleotide region of the labeled binding reagent, for example using a disulfide bond, such that it can be cleaved (reduced) and removed for successive imaging using labeled binding reagents incorporating the same dye. In one embodiment, the dyes for the labeled binding reagents are selected from among Cy5, BODIPY 630/650-X, LC Red 640, Alexa Fluor 633, BODIPY 650/665-X, Alexa Fluor 647, Alexa Fluor 660, Cyanine5.5, Alexa Fluor 680, Alexa Fluor 700 and Alexa Fluor 750.

In one embodiment, step (f) is accomplished by hyperspectral scanning of the specimen. In other embodiments, step (f) is accomplished by standard fluorescence imaging, light sheet imaging, or flow cytometry. In some embodiments, step (f) is accomplished by non-optical sensing methods such as mass spectrometry.

In one embodiment, the imaging at positions throughout the specimen to detect the labeled binding reagents and extent of labeling is obtained sequentially or simultaneously. In some embodiments, the specimen is imaged for all dyes at each location in the specimen simultaneously. In some embodiments, the specimen is imaged for each dye sequentially at each location in the specimen. In some embodiments, the specimen is incubated with all of the bivalent binding reagents and the subsequent incubating with the labeled binding reagents may be simultaneous or sequential, with, in some embodiments, imaging after each sequential incubation with each labeled binding reagent or a subset of the labeled binding reagents. Thus, in some embodiments, the imaging is performed batchwise to detect one or more dyes each scan. In some embodiments, after such partial imaging of the sample, the one or more labeled binding reagents are washed out of the specimen before the next one or more labeled binding reagents are incubated then imaged. In some embodiments, the washing out comprises removing the labeled binding reagents. In other embodiments, the dyes of the labeled binding reagents are quenched or otherwise made to not interfere with subsequent imaging of the same or different dyes.

In one embodiment, the data obtained from step (f) is converted to locations of particular cell types within the specimen using the correlating of step (g), which is based upon the relationships established in step (c). In one embodiment, step (c) is an encoding step, and step (g) is a decoding step. In one embodiment, the method used for coding in step (c) is used for decoding in step (g).

In one embodiment, the locations of the cell types within the specimen in step (h) are used diagnostically to identify, for example, a normal state, a disease state or the potential for a diseases state to develop, based upon the locations of particular cell types within the specimen.

In some embodiments, the incubating of the specimen with the bivalent binding reagents is performed before the incubating with the labeled binding reagents. In some embodiments, the incubating with the bivalent binding reagents is of longer duration than incubating with the labeled binding reagents. In some embodiments, the incubating with the bivalent binding reagents is performed at the same time as with the labeled binding reagents. In some embodiment the specimen is washed after incubating with the bivalent binding reagents and before the incubating with the labeled binding reagents. In some embodiments the labeled binding reagents are added after the bivalent binding reagents. In some embodiments the specimen is washed after incubation with the labeled binding reagents.

In some embodiments, the specimen after the incubating steps is imaged in a single scan. In some embodiments the specimen is imaged using multiple scans. In some embodiments, all dyes used in the labeled binding reagents are imaged at each specimen location at the same time. In some embodiments, a subset of dyes are imaged at each location at the same time. In some embodiments, one dye is imaged at each location. In some embodiments, imaging and incubations steps are repeated in a sequence where each step a different set of labeled binding reagents are incubated, excess is washed, and bound reagents are imaged. In some embodiments, the sequential or simultaneous incubation of the specimen with the bivalent binding reagents and the labeled binding reagents are independent of the sequential of simultaneous imaging. In some embodiments, the specimen is incubated with all of the bivalent binding reagents, but the incubating with the labeled binding reagents may be simultaneous or sequential, with, in some embodiments, imaging after each incubation with each labeled binding reagent.

In some embodiments, after each imaging of the sample, the one or more labeled binding reagents are washed out of the specimen before the next one or more labeled binding reagents are incubated then imaged. In some embodiments, the washing out comprises removing the labeled binding reagents. In some embodiments, the washing out comprises reducing a disulfide that is binding the dye to the labeled binding reagent, and washing the specimen.

In some embodiments, the imaging is low magnification imaging. In some embodiments, the specimen is cleared before incubation or imaging. In some embodiments, the specimen is embedded in a hydrogel before incubating or imaging.

In some embodiments, the specimen is brain. In some embodiments, the set of bivalent binding reagents for scanning brain cell types are comprise one or more of SEQ ID NOs:25-48. In some embodiments, the set of labeled binding reagents comprise one or more of SEQ ID NOs:75-98. In some embodiments, the set of bivalent binding reagents for scanning brain cell types comprise SEQ ID NOs:25-48 and the set of labeled binding reagents comprise SEQ ID NOs:75-98.

In some embodiments, the specimen is a whole organ. In some embodiments, the specimen is fresh, frozen, formalin preserved, alcohol preserved, a thin section, a thick section, a biopsy specimen or a previously formalin-fixed, paraffin embedded specimen. In some embodiments, the specimen is obtained from a patient, a healthy subject, a pathology specimen, a fossilized specimen, a frozen or cryogenically preserved specimen, an exhumed specimen or a mummified specimen.

In some embodiments, the bivalent binding reagent comprises an antibody or antigen-binding fragment.

In some embodiments, a bivalent binding reagent is provided selected from among SEQ ID NOs:25-48.

In some embodiments, a labeled binding reagent is provided selected from among SEQ ID NOs:75-98.

These and other aspects of the embodiments described herein will be provided in the ensuing descriptions of the drawings and detailed description of the embodiments.

### BRIEF DESCRIPTION OF THE DRAWINGS

Exemplary embodiments of the disclosure will be better understood and readily apparent to one of ordinary skill in the art from the following written description, by way of example only, and in conjunction with the drawings, in which:

FIG. 1 depicts a block diagram illustrating the concept of the method of the present disclosure.

FIG. 2 depicts an exemplary cell taxonomy based on transcriptome data.

FIGS. 3A, 3B, and 3C depict a mathematical definition of a discriminant projective non-negative matrix factorization (dPNMF) approach, wherein C signifies the number of classes, n_{c}, is the number of examples of class c, and S_{w} and S_{b} signify the within-class scatter and between-class scatter, respectively. Oher approaches such as nearest-neighbor algorithms are alternatives.

FIGS. 4A-4C illustrate how dimensionality-reduced FISH (dredFISH) directly measures the lower-dimensional representation of gene expression. FIG. 4A is a block matrix diagram of expression of three genes: G₁G₂G₃ (left matrix, “cell×gene”), the non-negative projection matrix (middle box, “Projection matrix”) and the resulting lower-dimensional representation of cell-1 and cell-2 in new basis B1 and B2 (right box, “Low-dimensional representation”). FIG. 4B shows experimentally, the cell by gene (“cell×gene”) matrix is simply the number of mRNAs for each gene G₁G₂G₃ (shown as lines of different density) from each gene that are expressed in each cell. The projection matrix is implemented by a pool of bivalent DNA oligo probes (bivalent binding reagents). Each probe (bivalent binding reagent) maps to a gene sequence (bottom part, molecular-marker binding region) and to a readout sequence (top part; labeled-binding-reagent binding region). If the weight is larger than 1, multiple molecular-marker binding region sequences are used in different bivalent binding reagents that bind to the same molecular marker. For example, the value for the first item in the matrix (G₁, B₁) is 3. It is implemented by including three oligos in the pool targeting different 25-mers sequences in gene G₁. The total number of oligos in the pool is therefore equal to the sum of the weights in the projection matrix. Using this design, staining cells is equivalent to performing matrix multiplications. The resulting composite readout in the sample is the number of bivalent binding reagents (probes) that map to the B₁ and B₂ labeled binding reagents (readout probes). The number of readout arms of each basis type (B₁ and B₂) in each cell is a sum of the products of the number of weights per gene times the number of RNA per gene. Thus, in total, cell-1 will have 7 B₁ (7=3*2+1*1+0*1) and 4 B₂ (4=0*2+2*1+2*1) whereas cell-2 will have 4 B₁ (4=3*1+1*1+0*3) and 8 B2 (8=0*1+2*1+2*3). These values are compared between experimental measurement and reference to allow classification of cells into types and reconstruction of gene expression. FIG. 4C depicts “dredFISH space”: dimensionality reduced representation of cellular transcriptional state. The dredFISH values of reference cells are calculated using scRNAseq data and compared to the directly measured values.

FIGS. 5A-5B depict cell type encoding methods. FIG. 5A depicts a PCA-basis cell type encoding. FIG. 5B depicts a dPNMF-basis cell type encoding. The advantages of dPNMF include the sparsity and non-negative weights.

FIG. 6 shows that dredFISH directly measures an approximate cellular transcriptional state. Twelve representative dredFISH experimental measurements (out of 24) are shown in one hemisphere of mouse brain coronal section (approx. 50,000 cells). Each of the basis measurements is based on weighted sums of different genes. As different cells express different set of genes, the overall pattern of dredFISH measurements vary spatially, layers, hippocampus, thalamus, and many more.

FIGS. 7A-7G depicts dredFISH based cell types inference matches known cell types. FIG. 7A shows UMAP embedding of harmonized scRNA-seq and dredFISH data shows the results of the iterative normalization procedure used as part of dredFISH analysis. Left panel is colored by technology whereas middle and right panels are colored by cell type. For the scRNAseq data the cell types were called by Allen Institute for Brain Science, cell types for dredFISH come from label transfer based on KNN classification. FIG. 7B shows the average transcriptional state in the projected dredFISH basis for all identified cell types. c. Boxplot shows Pearson correlation coefficients between scRNA-seq and dredFISH for every cell type in (FIG. 7B) across 24 bits. FIGS. 7D-F show the spatial distribution of cell types classified in supervised manner using scRNAseq reference data. The panels show classification at three different cell type resolutions. FIG. 7D shows Level-1 classifies cells into three: Glutamatergic, GABAergic, and non-neuronal. FIG. 7E shows Level-2 expands each of level-1 classes. The expansion of Glutamatergic neurons into five additional subtypes is shown. FIG. 7F shows the subtype DG/SUB/CA from level-2 is further classified into CA1-ProS, CA3 and DG types. Overall cells were classified into 44 distinct level-3 types. Only three are shown here for clarity. FIG. 7G shows ground truth data for comparison with FIGS. 9D-F. Top shows CA1-ProS, CA3, and DG neurons classified based on our MERFISH data. Bottom shows ISH from Allen Brain Atlas with marker genes for cortical layers (top row) and regions within the hippocampus that correspond to the CA1-ProS, CA3, and DG neuronal types.

FIGS. 8A-D show that dredFISH measurements enable common spatial transcriptomics data analysis tasks. FIG. 8A shows a Leiden graph-based cluster analysis of cells in regions outside of the reference scRNAseq (grayed out region) identified 61 putative cell types. FIG. 8B shows a region analysis using topic modeling divided the tissue into distinct anatomical regions (left) that qualitatively match Allen Brain Atlas (right). FIG. 8C shows reconstruction accuracy, defined as the explained variance of kNN regression normalized to total explained variance by PCA (x-axis). Reconstruction is poor for genes that do not match broad expression patterns (i.e. explain variance by PCA <0.2) but high for genes that are aligned with broad expression patterns. Reconstruction values >1 occur when kNN regression outperforms linear PCA. FIG. 8D shows examples of gene expression reconstructions compared to ISH data.

FIGS. 9A-9F depict a neural network probe design. FIG. 9A shows schematics of the statistical model. The input to the model is the cell by gene matrix X that has ˜10 k columns (genes). The first layer (fₑ) is the design matrix that maps genes to the readout basis. It is subjected to multiple constraints, i.e. all entries are non-negative, and regularization. The network simulates the effect of noise (Poisson for expression+dropout for loss of probes) and predicts cells types (C) and gene expression reconstruction (Xre) using layers fc and fg respectively. To allow the integration of multiple datasets in the reference, a discriminator is added that aims to determine the data sources. The added discriminator ensures that the identified projection is insensitive to batch effects in input data. FIG. 9B shows the classification accuracy of this model design based on cross-validation of reference dataset. The classification into Level-3 types (circles) was >95% accurate for SMART-seq (black) and 10× (orange) reference data. Interestingly, the new design performs well (>75% accuracy) when tasked with a more challenging task of classifying all known Level-5 sub-types defined by Allen Institute for Brain Science (388 subtypes). FIG. 9C is an example of neural network based design (left) compared to DPNMF design (right). The NN design is more balanced in the number of probes allocated to each basis (column) compared to existing DPNMF design. FIG. 9D shows that the new design addresses the issue of non-uniformity of measurements. DPNMF based design (blue) shows >3 orders of magnitude difference in expected intensity across the 24 basis measurements. The NN based design (orange) is uniform in its expected intensity. FIG. 9E shows the resulting average basis per cell type for both designs. Each row represents a known cell type and each column one of the 24 approximate basis calculated using reference data. FIG. 9F shows a Pearson correlation matrix of cell type signatures for the neural network design (left) and DPNMF design (right). The NN design has lower similarities between rows compared to DPNMF design as apparent in the Pearson correlation matrices. The low correlations between types is indicative of increased signature diversity that will increase classification accuracy.

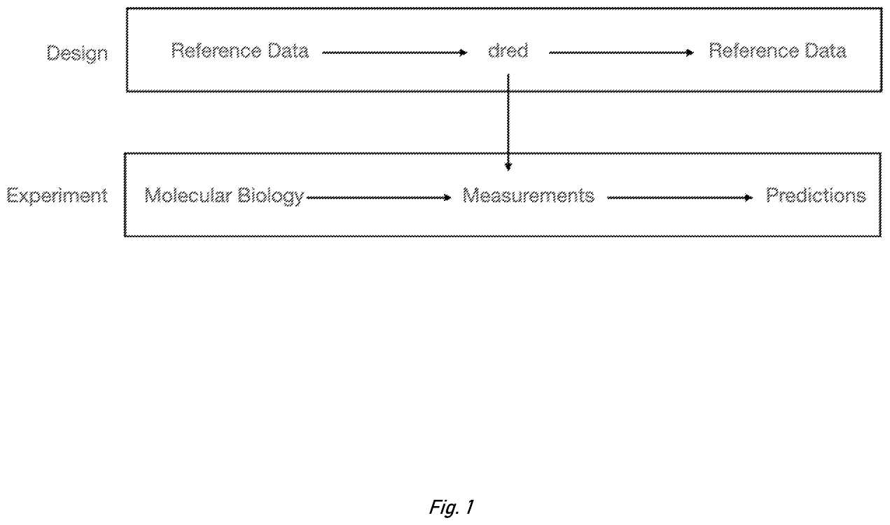

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings:

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

“Cell type” refers to a classification of biological cells using a classification system wherein at least two cells have at least one difference between them, and are thereby classified as different cell types, but typically refers to a taxonomy wherein the genotypic and/or phenotypic properties of a cell define its type, such properties including but not limited to (a) anatomical morphology; (b) apparent physiological function; (c) protein markers; (d) genetic markers; (e) developmental origins and taxonomy; (f) the organ and/or tissue and/or structure where the cell is typically found in an organism; (g) epigenetic states and markers; (h) electrophysiology; (i) cross-species homology; or (j) combinations of any of these factors. As used herein, cell type refers to a cell having properties different from any other cell as differentiated by the methods described herein. It will be readily understood to persons skilled in the art that any particular cell may have many possible valid “cell type” classifications according to various different heuristics and/or levels of specificity. For example, hepatic stellate cells could be classified as having “liver cell” cell type, or could be classified as having “neutrophin-expressing cell” cell type. (See C. Schachtrup et al., Hepatic stellate cells and astrocytes, CELL CYCLE, 2011 Jun. 1; 10(11: 1764-1771).

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif.

As used herein, the terms “protein,” “polypeptide,” and “peptide” refer to a molecule comprising amino acids joined via peptide bonds. In general, “peptide” is used to refer to a sequence of 20 or less amino acids and “polypeptide” is used to refer to a sequence of greater than 20 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest.

As used herein, the term “native” (or wild type) when used in reference to a protein refers to proteins encoded by the genome of a cell, tissue, or organism, other than one manipulated to produce synthetic proteins.

Abbreviations used herein for nucleotides shall adhere to industry standards as defined in WIPO Standard ST.25, Annex C, Appendix 2, Tables 1 & 2. Abbreviations used herein for the canonical proteinogenic amino acids adhere to industry standards as defined in WIPO Standard ST.25, Annex C, Appendix 2, Tables 3 & 4, and should be readily understood by persons having ordinary skill in the art. Amino acids abbreviated with the prefix D- refer to the D-enantiomer, but without any prefix shall be understood as referring to the L-enantiomer. As used herein, modified, uncommon, and non-proteinogenic amino acids shall be abbreviated as follows: Aad=2-aminoadipic acid; bAad=3-aminoadipic acid; Acpc=1-aminocyclopropanecarboxylic acid; bAla=β-alanine (i.e., β-aminoproprionic acid); Abu=2-aminobutyric acid; 4Abu=4-aminobutyric acid (i.e., piperidinic acid); Acp=6-aminocaproic acid; Ahe=2-aminoheptanoic acid; Aib=2-aminoisobutyric acid; bAib=3-aminoisobutyric acid; Apm=2-aminopimelic acid; Dbu=2,4-diaminobutyric acid; Des=desmosine; Dpm=2,2′-diaminoproprionic acid; Dpr=2,3-diaminoproprionic acid; EtGly=N-ethylglycine; EtAsn=N-ethylasparagine; Hse=homoserine (i.e., isothreonine); Hyl=hydroxylysine; aHyl=allo-hydroxylysine; 3Hyp=3-hydroxyproline; 4Hyp=4-hydroxyproline; Ide=isodesmosine; alle=allo-Isoleucine; MeGly=N-methylglycine (i.e., sarcosine); MeIle=N-methylisoleucine; MeLys=6-N-methyllysine; MeVal=N-methylvaline; Nva=norvaline; Nle=norleucine; and Orn=ornithine. Additionally, some alternative abbreviations for uncommon amino acids may be known to those having ordinary skill in the art, and may be readily understood from their context. For example, sometimes norleucine may be abbreviated as “norLeu” and homoserine may be abbreviated as “homoSer.”

A “sequence read” or “read” refers to data representing a sequence of monomer units (e.g., bases) that comprise a nucleic acid molecule (e.g., DNA, cDNA, RNAs including mRNAs, rRNAs, siRNAs, miRNAs and the like). The sequence read can be measured from a given molecule via a variety of techniques.

As used herein, a “fragment” refers to a nucleic acid molecule that is in a biological sample. Fragments can be referred to as long or short, e.g., fragments longer than 10 Kb (e.g. between 50 Kb and 100 Kb) can be referred to as long, and fragments shorter than 1,000 bases can be referred to as short. A long fragment can be broken up into short fragments, upon which sequencing is performed.

A “mate pair” or “mated reads” or “paired-end” can refer to any two reads from a same molecule (also referred to as two arms of a same read—arm reads) that are not fully overlapped (i.e., cover different parts of the molecule). Each of the two reads would be from different parts of the same molecule, e.g., from the two ends of the molecule. As another example, one read could be for one end of the molecule in the other read for a middle part of the molecule. As a genetic sequence can be ordered from beginning to end, a first read of a molecule can be identified as existing earlier in a genome than the second read of the molecule when the first read starts and/or ends before the start and/or end of the second read. More than two reads can be obtained for each molecule, where each read would be for a different part of the molecule. Usually there is a gap (mate gap) from about 100-10,000 bases of unread sequence between two reads. Examples of mate gaps include 500+/−200 bases and 1000+/−300 bases.

“Mapping” or “aligning” refers to a process which relates a read (or a pair of reads, e.g., of a mate pair) to zero, one, or more locations in a reference sequence to which the read is similar, e.g., by matching the instantiated arm read to one or more keys within an index corresponding to a location within a reference.

As used herein, an “allele” corresponds to one or more nucleotides (which may occur as a substitution or an insertion) or a deletion of one or more nucleotides. A “locus” corresponds to a location in a genome. For example, a locus can be a single base or a sequential series of bases. The term “genomic position” can refer to a particular nucleotide position in a genome or a contiguous block of nucleotide positions. A “heterozygous locus” (also called a “het”) is a location in a reference genome or a specific genome of the organism being mapped, where the copies of a chromosome do not have a same allele (e.g. a single nucleotide or a collection of nucleotides). A “het” can be a single-nucleotide polymorphism (SNP) when the locus is one nucleotide that has different alleles. A “het” can also be a location where there is an insertion or a deletion (collectively referred to as an “indel”) of one or more nucleotides or one or more tandem repeats. A single nucleotide variation (SNV) corresponds to a genomic position having a nucleotide that differs from a reference genome for a particular person. An SNV can be homozygous for a person if there is only one nucleotide at the position, and heterozygous if there are two alleles at the position. A heterozygous SNV is a het. SNP and SNV are used interchangeably herein.

Sequencing refers to the determination of intensity values corresponding to positions of one or more nucleic acids. The “intensity values” can be any signal, e.g., electrical or electromagnetic radiation, such as visible light. There can be one intensity value per base, multiple intensity values per base, or fewer intensity values than there are bases. Also, an intensity value can be for a particular position, or an intensity value can be for multiple positions of a nucleic acid. Intensity values can be restricted to predetermined values (e.g., binary or integers in a decimal numeral system), or can have continuous values.

A “sequencing process” or “sequencing run” refers to the determination of intensity values corresponding to positions of one or more nucleic acids as a batch. For example, when the sequencing involves imaging biochemical reactions of nucleic acids on a substrate, the resulting intensity values are obtained during the same sequencing run. Intensity values of nucleic acids for a different substrate would appear in different sequencing runs. A nucleic acid of a first sequencing run would not be involved in a second sequencing run (e.g., not included in a same image).

An “assumed sequence” corresponds to the sequence that is believed to be accurate. The determination may be inaccurate, but the training assumes it is accurate. The assumed sequence can be determined in a variety of ways, e.g., as described herein. An assumed sequence can include no calls, and thus an assumed sequence can have open positions between called positions.

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “external loop portion” refers to the portion of transmembrane protein that is positioned between two membrane-spanning portions of the transmembrane protein and projects outside of the membrane of a cell.

As used herein, the term “tail portion” refers to refers to an n-terminal or c-terminal portion of a transmembrane protein that terminates in the inside (“internal tail portion”) or outside (“external tail portion”) of the cell membrane.

As used herein, the term “secreted protein” refers to a protein that is secreted from a cell.

As used herein, the term “membrane motif” refers to an amino acid sequence that encodes a motif not a canonical transmembrane domain but which would be expected by its function deduced in relation to other similar proteins to be located in a cell membrane, such as those listed in the publicly available psortb database.

As used herein, the term “consensus protease cleavage site” refers to an amino acid sequence that is recognized by a protease such as trypsin or pepsin.

As used herein, the term “affinity” refers to a measure of the strength of binding between two members of a binding pair, for example, an antibody and an epitope and an epitope and an MHC-I or II haplotype.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., IMMUNOL. TODAY, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In other embodiments, suitable monoclonal antibodies, including recombinant chimeric monoclonal antibodies and chimeric monoclonal antibody fusion proteins are prepared as described herein.

According to the disclosure, techniques described for the production of single chain antibodies (U.S. Pat. No. 4,946,778) can be adapted to produce specific single chain antibodies as desired. An additional embodiment of the disclosure utilizes the techniques known in the art for the construction of Fab expression libraries (Huse et al., SCIENCE, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) and the like.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), solid-state drives (SSD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

A “machine-learning model” (also referred to as a model) refers to techniques that predict output base calls based on known results (training data). The known results can be an assumed sequence, which is assumed to be correct. As the model attempts to predict the results of the training data, the machine learning can be supervised learning, where the supervision comes from the training data.

A “base call” is a determination of a base at a position in a nucleic acid. A base call can be a no-call or a specified base. A base call can be made independently or as part of a combination of specified base (e.g., A/T), which can be for a same genomic position (e.g., if respective scores are close to each other) or for multiple positions. A “score” output from a machine-learning model can be used to determine a base call at a position. For example, a score can be provided for each of the bases. The determination of the base call based on the scores can be considered part of the model. Some models can provide a score, where the scores are used by a later process. Examples of a score can be a probability or a possibility. The probability scores for each of the bases would sum to a fixed number, i.e., one. The possibility scores are not required to sum to the fixed number. Each possibility score can be constrained to be between 0 and 1. The possibility scores could sum to 1, particularly if a model is trained well.

As used herein, the term “neural network” refers to various configurations of classifiers used in machine learning, including multilayered perceptrons with one or more hidden layers, support vector machines and dynamic Bayesian networks. These methods share in common the ability to be trained, the quality of their training evaluated and their ability to make either categorical classifications or of continuous numbers in a regression mode.

As used herein, the term “principal component analysis” refers to a mathematical process which reduces the dimensionality of a set of data (Wold, S., Sjorstrom, M., & Eriksson, L., Chemometrics and Intelligent Laboratory Systems 2001. 58: 109-130; Multivariate and Megavariate Data Analysis Basic Principles and Applications (Parts I&II) by L. Eriksson, E. Johansson, N. Kettaneh-Wold, & J. Trygg, 2006 2^{nd} Ed. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements.

The terms “dimensionality reduction” or “dimension reduction” (sometimes abbreviated “dred”) refers to the process of reducing the number of variables or features under consideration, via obtaining a set of “uncorrelated” principal variables.

As used herein, the term “vector” when used in relation to a computer algorithm or the present disclosure, refers to a numerical-array representation of an object or feature, such as, e.g., a nucleic acid or a protein, generated such that an algorithm may perform processing and statistical analysis. (For instance, a “COLOR” vector may be defined as a numerical-array representation of the amount of red, green, and blue there is in the chosen color. The COLOR vector could be defined numerically as COLOR=[R, G, B] where R, G, and B are numeric representations of the amplitude of red, green, and blue present.)

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

The terms “bacteria” and “bacterium” refer to prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., C V Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red. In some embodiments, the bacteria are those capable of causing disease (pathogens) and those that cause product degradation or spoilage.

The terms “fluorescent label”, “fluorescent tag”, and “fluorescent probe” describe a molecule or molecules that attach chemically to assist in the detection of a biomolecule such as, e.g., a protein, antibody, nucleic acid polymer, amino acid, and/or lipid. Fluorescent labels may comprise fluorescent proteins, such as, e.g., blue fluorescent proteins, cyan fluorescent proteins, green fluorescent proteins, red fluorescent proteins, and yellow fluorescent proteins. Exemplary fluorescent labels include, but are in no way limited to, Sirius, Azurite, EBFP, EBFP2, FCFP, Cerulean, CyPet, SCFP, eGFP, Emerald, Superfolder avGFP, T-Sapphire, RFP, mCherry, mOrange, mRaspberry, mRuby, FusionRed, EYFP, Topaz, Venus, Citrine, YPet, SYFP, and mAmetrine. Fluorescent labels may comprise dyes, such as the blue-fluorescent DNA stain 4′,6-diamidino-2-phenylindole (DAPI). Fluorescent labels may also comprise other fluorescent biomolecule stains such as, e.g., BODIPY lipid conjugates.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The human can be any human of any age. In an embodiment, the human is an adult. In another embodiment, the human is a child. The human can be male, female, pregnant, middle-aged, adolescent, or elderly.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is male human or a female human.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

The following descriptions of each step in the process are intended as guidance on means for carrying out the step in relationship to other steps in the process, and are not intending to be limiting. One of skill in the art will be able to modify the steps without deviating from the spirit of the disclosure.

Although significant advances in the development of highly multiplexed spatial measurements of tissues has occurred over recent years, including, e.g., combinatorial FISH using MERFISH and seqFISH+, highly multiplexed (≥30) immunofluorescence measurements using metal-conjugated antibodies, repeated cycles of immunofluorescence imaging and signal removal, in situ sequencing, and spatial DNA barcoding, and such highly multiplexed spatial biology is rapidly becoming a “standard” approach, analogous to the rapid adoption of single-cell sequencing technologies, Existing spatial transcriptomics approaches are ill-suited for volumetric three-dimensional measurements of RNA in whole organs and complex two-dimensional specimens. The limitations stem from inherent technical requirements that limit their applicability to very thin 2D sections. In the case of spatial DNA barcoding, capture of RNA necessitates thin sections with spatially defined capture bins. Analysis of the whole mouse brain will require approximately 10³ sections, 10⁹ spatial bins, requiring an unrealistic number of 10¹⁴ sequencing reads. Combinatorial FISH can only be done in thin sections due to the need for a high spatial resolution to allow subdiffraction single-molecule detection. Targeted approaches that focus on a smaller number of genes can speed acquisition. However, (i) the requirement for thin sections is independent of the number of measured genes. (ii) using a targeted approach is likely to never produce the “final” map, as it is very likely that current transcriptional definitions of cells might change in the future, especially when one considers more developmental stages, disease models, etc. Therefore, technologies that leverage prior cell type definitions need to be fast in order to allow recreating the atlas with any update to cell type taxonomy. Existing spatial transcriptomics approaches lack the scale to make whole organ cell type mapping a routine experiment.

The present disclosure implements a novel and unexpected approach to cell-type mapping that overcomes significant problems with existing methods and systems. The present disclosure is based on a new fluorescence in situ hybridization (FISH) variant, dubbed dimensionality-reduced FISH (“dredFISH”). DredFISH combines direct measurement(s) of low-dimensional representation(s) of single-cell transcriptomics with a supervised machine learning algorithm to spatially map cell types, bypassing any need to measure expression of single genes.

In one embodiment, dredFISH leverages existing single-cell RNA sequence (scRNAseq) data from over 370 brain cell types and a supervised basis identification algorithm known as discriminant Projective Non-Negative Matrix Factorization (dPNMF), in one embodiment, to design aggregate measurements based on the non-negative weighted sums of the expression(s) of thousands of genes optimized to preserve distinguishable cell-type information. DredFISH experimentally implements these weights through an oligonucleotide (oligo) design to allow for direct measurement of the low-dimensional approximation of cells' transcriptional state, thus leapfrogging the need for direct gene expression measurements. Using a supervised algorithm trained on labeled scRNAseq data, one may classify cells into their types based on their experimentally measured reduced-dimensionality representation(s). Such methods may be applied to other specimens, cell types, expression of other markers, and/or other reagents, in order to perform similar classifications.

Further, light-sheet imaging of cleared brain tissue labeled with dredFISH probes will be able to provide the throughput needed for whole brain mapping at single-cell resolutions. Current sequential hybridization techniques are problematic in large volumes due, among other issues, to probe penetration. In some embodiments, dredFISH may utilize a hyperspectral light-sheet microscope leveraging newer organic polymer dyes, allowing simultaneous imaging and measurement of numerous fluorophores through all positions in a specimen.

As described herein, the present disclosure provides for the identification of the locations within a specimen of specific cell types. However, such identification is not based on a “1:1” correlation between the cell type and its location as would be determined by conventional cell staining or even more advanced methods using immunocytochemistry or in-situ hybridization, where the specific position of a cell in a specimen is based on a detectable property (e.g., antibody binding, nucleic acid hybridization) at a location; such methods for identifying locations of numerous cells types in a large specimen are tedious, time consuming and often unnecessary in order to yield the desired information. In contrast, the methods described herein provide a higher level cell type classification within the specimen based on a plurality of properties of each cell type (e.g., receptor expression, nucleic acid expression), a plurality of specific reagents that bind to the certain receptors or nucleic acids expressed by each cell type (referred to herein as bivalent binding reagents or encoder probes), a plurality of detectably-labeled specific reagents that bind to the bivalent binding reagents (herein referred to as labeled binding reagents) a means (e.g., imaging) to readily identify the locations of the labeled binding reagents in all locations within the specimen, and an in silico designed selection of properties, reagents and labels that differentiates among the cell types and readily provides cell type location. As described herein, it has been found that a limited number of detectable dyes, cell detection reagents, and the intensity of detectability of each reagent, can provide the cell location information without locating every differentially expressed cell specific receptor or nucleic acid amongst a plurality of cell types can be used to generate cell type locations.

The methods disclosed herein are useful beyond any specific examples of nucleic acid or protein detection (using, e.g., complementary nucleic acids or antibodies, respectively), for any cellular component for which a tag or tags may be employed following the guidance here to detect the component(s) of interest to identify and differentiate cell types in the specimen. Furthermore, different types of tags (e.g., both antibodies and nucleic acids) may be used together in the practice of the methods disclosed herein for the purposes described herein. Thus, while nucleic acid-based tags are employed in certain examples herein, for tagging nucleic acids in cells, the disclosure is not so limiting to any particular type of tag or any particular use of a single type of tag in a particular method. Non-limiting examples of other molecular markers include metabolites, lipids, carbohydrates including polysaccharides, glycolipids, vitamins, fatty acids, co-factors, pigments, metals, or any other biochemicals or compounds, organic or inorganic, found within a biological system. Tags useful for their detection in accordance with the teaching herein include but are not limited to antibodies and antigen-binding fragments thereof, ligands, lectins, receptors, chelators, etc.

The following descriptions of the steps of the method are exemplary and non-limiting. Variations that achieve the same or similar outcomes are fully embraced herein.

Selecting Cell Types within the Specimen (Step a)

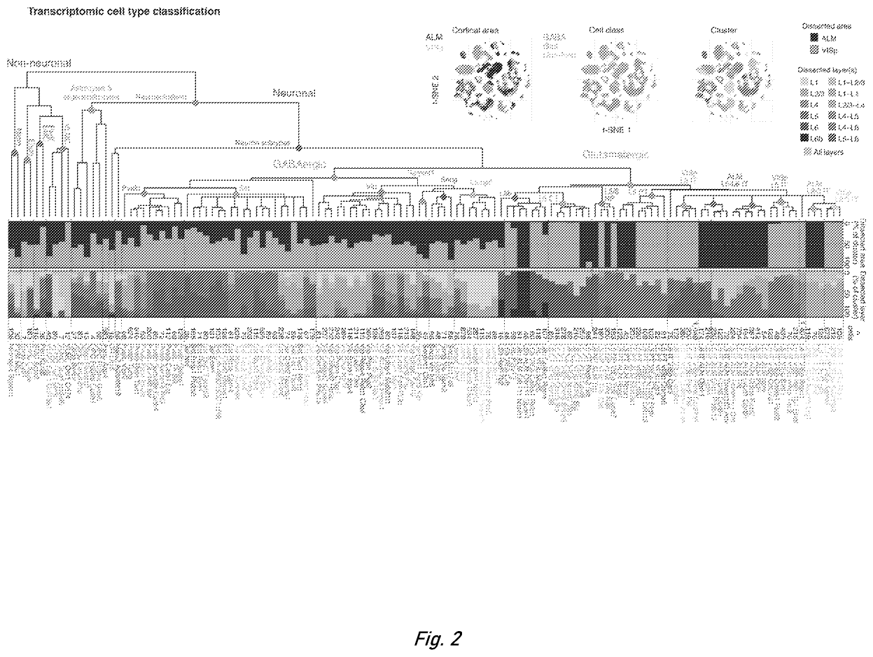

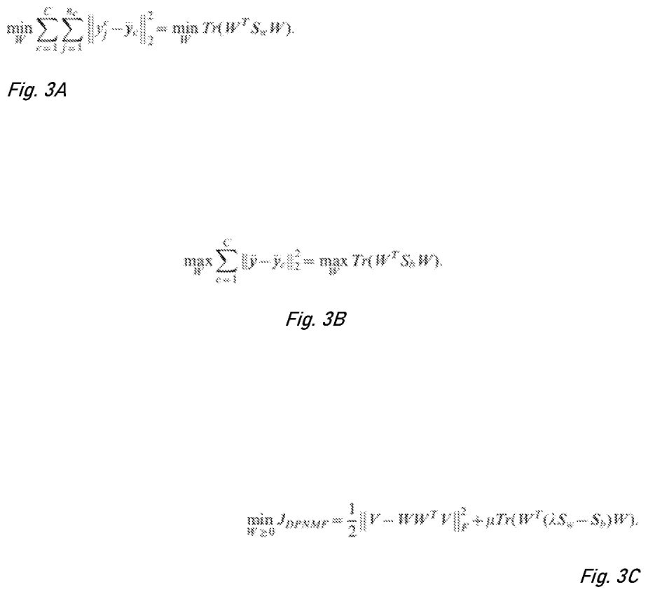

The cell types to be located within a specimen is guided by the information desired to be obtained by locating the positions, numbers, distribution, topography, contact zones, organization, purity, shape, and/or other characteristics of such cell types within the specimen and/or relationships to other cellular, tissue or organ structures. By way of example, the distribution of cancer cells in stroma from a solid tumor biopsy, or the distribution of astrocytes and neuronal cells in the hippocampus, may be diagnostic for cancer invasiveness or neurodegeneration, respectively. Moreover, mapping of cell types using the methods disclosed herein using a normal cellular sample, specimen, tissue or organ may provide information such as what comprises a normal (e.g., healthy) cell type distribution against which to compare pathological or suspected pathological specimens. Changes in cell type distributions over time may provide methods for determining chronological or biological age from a specimen. For example, FIG. 2 lists cell types in the brain, and shows the classification of these cell types in different categories and subcategories. While the skilled artisan may readily be cognizant of the types of cells in a particular biological sample of interest in localizing, such information on cell type makeup of organs and tissues in numerous animal species is available in the literature. Furthermore, the cell type makeup in pathological specimens is also available in the literature; methods as described here may add further diagnostically and/or therapeutically useful information to benefit patients.

It should be noted that specimens useful for the purposes herein may be fresh, frozen, formalin preserved, alcohol preserved, thin sections, thick sections, biopsy specimens, formalin-fixed, paraffin embedded, by way of non-limiting examples. Such specimens may come from patients, healthy subjects, pathology specimens, fossilized specimens, cryogenically preserved specimens, exhumed specimens, mummified specimens, etc. Moreover, such specimens may be embedded in a hydrogel matrix (e.g., polyacrylamide), may be cleared, may be expanded, or any combination of the above.

### Selecting Cell Type Molecular Markers (Step b)

The disclosure herein is based on identifying locations of cells relying on detectable expression of molecular markers on each of those cells types. Such markers may be unique to a particular cell type, or the same markers can be expressed in different amounts, absolutely or relative to one or more other markers, among a number of different cell types. As noted herein, the subsequent steps in which reagents are designed to optimally distinguish among cells types based on expression of such markers and may inform the selection of the markers to be used for the identification, such that steps (b) and (c) are interrelated, and the order they are carried out may be reversed or iterative.

The molecular markers of the selected cell types from step (a) may be identified from the literature. For example, the identity and levels of expression of cell surface markers among the numerous types of brain cells is known from the literature. Identities of markers expressed on or by numerous cell types in tissues and organs of numerous animal species are an expanding part of the scientific literature.

In one embodiment, the molecular markers are nucleic acid polymers. In a preferred embodiment, the nucleic acid polymers are RNA. In an alternative embodiment, the molecular markers are protein, which may be any cell-surface protein, receptor, transcription factor, antibody, or a combination thereof. In other embodiments, the molecular markers are metabolites, lipids, carbohydrates including polysaccharides, glycolipids, vitamins, fatty acids, co-factors, pigments, metals, or any other biochemicals or compounds, organic or inorganic, or any combination thereof. In some embodiments, as described further below, a single type of tag (e.g., antibody) is used to detect one or more markers. In some embodiments, two or more types of tags are used (e.g., nucleic acids and antibodies) to detect one or more types of markers. In some embodiments, two or more tags are used to detect two or more types or markers. The disclosure is not limited by the number of different types of tags or the number of types of markers identified using the methods herein.

### Establishing a Relationship (Step c)

The present disclosure comprises a computational method for determining how to optimally label cells in a biological sample such that each cell type of interest is distinguishable from each other cell types in the biological sample using a limited number of labels and using a number of probes of molecular markers, based on relative levels of marker expression among cell types of interest to be differentiated from each other. In one embodiment of the present method, up to hundreds of cell types may be reliably distinguished in a tissue sample using fewer than thirty detectable labels. Such discrimination among cell types is provided by the computational-aided selection (encoding) of binding reagents (e.g., bivalent binding reagents and labeled binding reagents) that detect those markers, the extent of detectability of each labeled binding reagent, the ability and efficiency of an imaging system to identify at each location in the specimen the labels, and the computation methods to decode the relative marker expression information back into specific cell types and locations. In some embodiments, step (c) is a machine learning based process. In some embodiments, step (c) is a dimensionality reduction process. In some embodiments, step (c) is global optimization heuristic such as simulated annealing or genetic algorithm.

In silico methods for designing the reagent set based on the above information are available in any number of formats, such as but not limited to algorithms including machine learning algorithms. Non-limiting examples of such algorithms include recursive partitioning, discernment projection non-negative matrix factorization (dPNMF), among others.

Typically, this step identifies and implements a lower-dimensional representation of gene expression followed by additional statistical learning steps that assign labels to cells in the lower dimensional space. One popular dimensionality reduction scheme is principal components analysis (PCA) that is often used to create a representation of gene expression data using the first 20-50 components. Therefore, in practice, cell type classification is not occurring in the original gene expression space rather in a dimensionality reduced space that captures enough information to accurately classify cells into types. In other words, the read-out used for cell type classification are not individual genes but, in the case of PCA, a small number of linear weighted sums of gene expression levels. Using statistical methods such as PCA or dPNMF, it is possible to avoid the need for individual gene expression measurements, circumventing the inherent challenges faced by spatial transcriptomics.

There are several dimensionality reduction methods that are typically applied to single-cell RNA expression data, the most popular of which are PCA and dPNMF. Other suitable methods may include those described below:

Recursive partitioning. In one embodiment, all cell types of interest in being located within the specimen are organized into a binary classification tree. The tree can be learned directly from observations determining among those specific cell types the known extent of expression or lack thereof of a plurality of known molecular markers of each specific cell type, using hierarchical clustering or using prior knowledge. FIG. 2 depicts such a tree of brain cell types from Yuste et al., 2020 (ibid.) Provided with this cell-type tree (i.e., cell taxonomy), the following operations are performed:
(1) Assign 1/0 labels to all cell types based on where their cell type is in relation to the initial split. For example, all cells with types that are on the left side of the split will get “1” all the cells that have type on the right will get “0”. In the brain example above, the first split is to “non-neurons” and “neurons” and all cells will be labeled accordingly.
(2) Fit a regularized logistic regression where the dependent variable is the “1”/“0” assigned above, and the independent variables are the abundances of all markers. The results of the fit are the coefficients and threshold. The coefficients are the weights for each marker such that when all the abundance of each marker are multiplied by these coefficients a maximal separation is obtained between the “0” and “1” class. The threshold is a value where above it the product of the coefficients times the abundance should be mapped to “1” and below it to “0”. The regularization is provided to enforce that all the weights are non-negative.
(3) Recursively traverse the tree, and at each step perform an analysis task similar to what was done for the root. Label all cells that are below the left side of the branch as “1” and the others as “0”, fit a logistic regression and obtain the same coefficients and threshold for each of the splits in the tree. Again, use regularization to ensure that all coefficients are non-negative.
(4) Use the weight coefficients to create an encoding. The number of fluorophores needed is the depth of the cell type tree. For each of these fluorophores, a staining is designed such that the number of fluorophore molecules that bind that marker is the value of the coefficient learned through the logistic regression.

Discernment projection non-negative matrix factorization (dPNMF). Another suitable method is a linear algebra method for basis identification called dPNMF, that is supervised and balances supervised label encoding and overall signal reconstruction. The dPNMF methodology has not yet been used previously in biological applications and has a key advantage over other basis identification methods as it identifies sparse representations that can be reapplied on unseen data. The method described in Guan et al., PLoS ONE, 2013 Dec. 20; 8(12):e83291, provides a means for learning from the data a dimensionality-reduced representation that can be obtained using only non-negative weights.

Artificial Neural Network. Another suitable method is the use of an artificial neural network with multiple layers. Each layer of the network implements two mathematical operations, linear matrix multiplication and a non-linear operation. The network is trained in a supervised manner to classify cells into their known types. To use supervised learning as a design step, we simply restrict the weight of the first operation (linear matrix multiplication) to have non-negative weights and add other restrictions related to the number of overall probes used to enforce sparsity. Converting the learned weights of the first layer to bivalent reagents that maps between markers and readout probes is done by multiplying this matrix by a constant and rounding to the closet integer. The resulting values are used to determine the number of bivalent probes that map each molecular marker to a specific readout probe.

The encoding step establishing the relationship between molecular markets and cell types, is achieved by discriminant projection non-negative matrix factorization (dPNMF) as follows:
(1) Fit dPNMF model to the training data.
(2) Fitting a classifier to one class per cell type. The results of the fit is a weight matrix that maps between the markers to a dimensionality-reduced space in a way that maximizes the information in that space about original marker abundance and cell type label.
(3) Implement the weights by creating a staining profile where the number of fluorophore molecules per marker approximate the learned non-negative weight.

In some embodiments, the classifier of step (ii) is a Naïve Bayesian classifier. In some embodiments, the classifier is a machine learning classifier. In some embodiments, the machine learning classifier is the K-nearest neighbors algorithm (KNN).

Thus, step (c) will provide the basis for the design of the bivalent binding reagents for use with a particular type of specimen. Based upon the weights assigned to each marker, bivalent binding reagents are prepared that may recognize multiple different sequences on a marker (a complementary nucleic acid for RNA markers; in the case of antibody-based reagents, they may recognize different epitopes on the same protein marker). Bivalent binding reagents that recognize different regions on the same marker and binding to the same labeled binding reagent thus provide the corresponding weight of that marker in the projection matrix, read onto the low-dimensional representation.

The foregoing methods for establishing the relationship, also referred to herein as encoding, may be practiced by other methods and the disclosure is not limited to any particular method. Furthermore, such establishing steps provided for a particular tissue or any other biological sample type may then be used for any other specimen of the same tissue or biological sample type, such that these steps need to be performed only once per sample type. In a non-limiting example, information for carrying out such steps may be stored and subsequently retrieved and used for processing additional specimens, including having the reagents described in steps (d) already prepared and ready for use, such that rapid processing and analysis of cell types locations in incoming tissues from a biopsy specimen or tumor resection, can be performed quickly for guiding drug therapy, further surgery, or both. Specialized reagents for detecting rare, abnormal, diseased or aberrant molecular marker expression may also be provided for diagnostic purposes.

### Preparing Bivalent Binding Reagents and Labeled Binding Reagents (Step d)

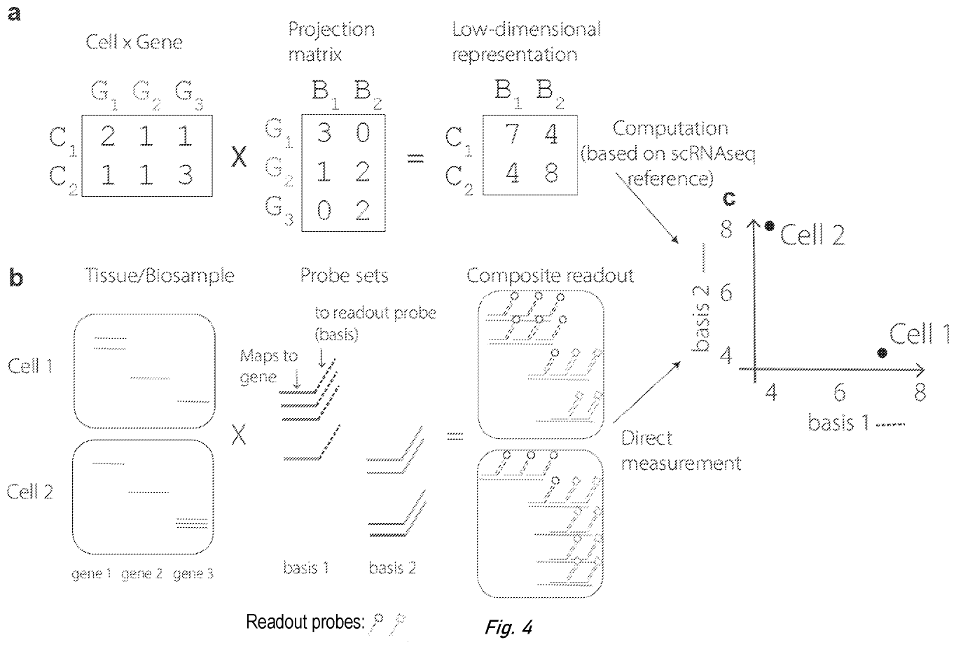

Provided with these encoded machine-learned data, which provides the weights to guide the design of the binding reagents, a staining protocol is created wherein the number of unique fluorophores equals the depth of the taxonomic tree, i.e., a vector of values, and the number of fluorophore molecules per binding reagent molecule the coefficient learned through the logistic regression. Bivalent binding reagents are prepared that bind to each molecular marker to be identified in the method; as noted, in some cases different bivalent binding reagents bind to multiple sites on a marker. Each molecular-marker-specific bivalent binding reagent comprises two parts, one that binds to the molecular marker, and another part that is bound by a labeled binding reagent. This probe sets design is shown in FIG. 4B, referring to the part of the bivalent binding reagent that maps to gene (the molecular-marker binding region, a region on a RNA marker whose presence and level characterized one cell type to be identified) and the other part binds to readout probe (labeled binding reagent). In one embodiment wherein the bivalent binding reagents are oligonucleotides, the part that maps to the gene is the part that hybridizes to the RNA marker on the cell. In this embodiment, the other part of the bivalent binding reagent, the part that binds to the readout probe, is designed to hybridize to a labeled oligo (e.g., fluorescently labeled oligonucleotide). Examples of designs of such probes are provided in subsequent examples. As noted below, the design of the set of bivalent binding reagents for a particular type of specimen may call for multiple bivalent binding reagents that bind to different parts of the same molecular target, each such bivalent binding reagent binding to the same labeled binding reagent.

Thus, in experimentally determining the design of the reagents for a particular specimen and cell types therein, a simplified design and guidance for preparing reagents is shown in FIGS. 4A-4C. In this example, the relative expression levels of three genes (RNAs) G₁, G₂ and G₃ are different between cell-1 (C₁) and cell-2 (C₂): cell-1 has twice the level of G₁; both cells have equal levels of G₂, and G₃ is expressed three times higher in cell-2 than cell-1. These differences form the basis for generating the low-dimensional representation, shown computationally in FIG. 4A, and experimentally in FIG. 4B, where the cells types can be clearly distinguished (FIG. 4C) using the probe sets (bivalent binding reagents and labeled binding reagents [readout probe]) shown. In this example, bivalent binding reagents that bind to G₁ are prepared that map to different parts of G₁ (these G₁-directed bivalent binding reagents have different molecular-marker binding regions that bind to different sequences of G₁, but have the same labeled-binding-reagent-binding regions). Thus, only two different labeled-binding-reagent-binging regions are needed for the bivalent binding reagents in this example. The total number of oligos in the pool is therefore equal to the sum of the weights in the projection matrix.

By way of further description of the example in FIG. 4, based on the block matrix diagram of expression of three genes: G₁G₂G₃ (left matrix, “cell×gene”), the non-negative projection matrix (middle box, “Projection matrix”) and the resulting lower-dimensional representation of cell-1 and cell-2 in new basis B₁ and B₂ (right box, “Low-dimensional representation”). The low-dimensional representation of a cell-gene the cell by gene (“cell×gene”) matrix is simply the number of mRNAs for each gene G₁G₂G₃ (shown with different density lines) from each gene that are expressed in each cell. The projection matrix is implemented by a pool of bivalent DNA oligo probes (herein called bivalent binding reagents), each probe mapping to a gene sequence (bottom part; herein called the molecular-marker binding region) and to a readout sequence (top part; herein called the labeled binding reagent binding region). If the weight is larger than 1, multiple gene sequences are used (i.e., different bivalent binding reagents recognize different regions on the same marker). For example, the value for the first item in the matrix (G₁, B₁) is 3. It is implemented by including three oligos in the pool targeting three different 25-mers sequences in gene G₁. As noted above, the total number of oligos in the pool is therefore equal to the sum of the weights in the projection matrix. Using this design, staining cells is equivalent to doing matrix multiplications.

The resulting composite readout in the sample is the number of probes that map to B₁ and B₂ readout probes. The number of readout arms of each basis type (B₁ and B₂) in each cell is a sum of the products of the number of weights per gene times the number of RNA per gene. Thus, in total, cell-1 will have 7 B₁ (7=3*2+1*1+0*1) and 4 B₂ (4=0*2+2*1+2*1) whereas cell-2 will have 4 B₁ (4=3*1+1*1+0*3) and 8 B2 (8=0*1+2*1+2*3). These values are compared between experimental measurement and reference to allow classification of cells into types and reconstruction of gene expression.

FIG. 4C depicts “dredFISH space”: dimensionality reduced representation of cellular transcriptional state. The dredFISH values of reference cells are calculated using scRNAseq data and compared to the directly measured values.

The bivalent binding reagents disclosed herein comprise a molecular-marker binding region and at least one labeled-binding-reagent binding region. Such reagents may be prepared by any methods known in the art. In an example herein, oligonucleotide reagents are prepared and used to carry out dredFISH.

Oligonucleotide-based bivalent binding reagents comprise at least one molecular-marker binding region nucleic acid sequence, and at least one labeled-binding-reagent binding region nucleic acid sequence. In some embodiments a bivalent binding reagent may comprise two labeled-binding-reagent binding sequences, binding the same or different labeled binding reagents. In some embodiments a bivalent binding reagent may comprise three labeled-binding-reagent binding sequences. In some embodiments a bivalent binding reagent may comprise more than three labeled-binding-reagent binding sequences. Such plurality of labeled-binding-reagent binding sequences on a bivalent binding reagent may bind one or more of the same, or two different, or two of the same and one different, or three all different labeled binding reagents, or any other combination thereof. Such number of labeled-binding-reagent binding sequences will be provided in the calculations from steps a, b and c as described herein.

For purposes of amplifying the bivalent binding sequences, amplification sequences may be include in the bivalent binding reagents, which may be retained or removed before use in the staining methods disclosed herein. In some embodiments, a forward and reverse amplification nucleic acid sequence are included in the bivalent binding reagent. In some embodiment, the forward amplification sequence is provided at the 5′ end of the bivalent binding reagent, and the reverse amplification sequence at the 3′end.

In some embodiments, the bivalent binding sequence may comprise, from 5′ to 3′, a forward amplification sequence, a molecular-marker binding sequence, a labeled-binding-reagent binding sequence, and a reverse amplification sequence. In some embodiments the aforementioned sequence is cleaved to a, from 5′ to 3′, a molecular-marker binding sequence and a labeled-binding-reagent binding sequence.

In some embodiments, the bivalent binding sequence may comprise, from 5′ to 3′, a forward amplification sequence, a labeled-binding-reagent binding sequence, a molecular-marker binding sequence, and a reverse amplification sequence. In some embodiments the aforementioned sequence is cleaved to a, from 5′ to 3′, a labeled-binding-reagent binding sequence and a molecular-marker binding sequence.

In some embodiments, the bivalent binding sequence may comprise, from 5′ to 3′, a forward amplification sequence, a labeled-binding-reagent binding sequence, a labeled-binding-reagent binding sequence, a molecular-marker binding sequence, and a reverse amplification sequence. In some embodiments the aforementioned sequence is cleaved to a, from 5′ to 3′, a labeled-binding-reagent binding sequence, a labeled-binding-reagent-binding sequence, and a molecular-marker binding sequence.

In some embodiments, the bivalent binding sequence may comprise, from 5′ to 3′, a forward amplification sequence, a labeled-binding-reagent binding sequence, a labeled-binding-reagent binding sequence, a molecular-marker binding sequence, a labeled-binding-reagent binding region, and a reverse amplification sequence. In some embodiments the aforementioned sequence is cleaved to a, from 5′ to 3′, a labeled-binding-reagent binding sequence, a labeled-binding-reagent-binding sequence, a molecular-marker binding sequence, and a labeled-binding-reagent binding sequence.

In some embodiments, the bivalent binding sequence may comprise, from 5′ to 3′, a forward amplification sequence, a labeled-binding-reagent binding sequence, a molecular-marker binding sequence, a labeled-binding-reagent binding region, a labeled-binding-reagent binding sequence, and a reverse amplification sequence. In some embodiments the aforementioned sequence is cleaved to a, from 5′ to 3′, a labeled-binding-reagent binding sequence, a molecular-marker binding sequence, a labeled-binding-reagent-binding sequence, and a labeled-binding-reagent binding sequence.

In any of the bivalent binding reagents disclosed herein, additional nucleotides (e.g., A, C, G, T) may be provided as spacers between the aforementioned regions, such as one or more A.

The foregoing examples of arrangements of the components of a bivalent binding reagent are merely exemplary and alternate designs of the reagents are embraced herein without deviating from the intent of the disclosure. Table 1 sets forth a subset of the bivalent binding sequences used in the brain cell type scanning example herein, said sequences comprise, from 5′ to 3′, a forward amplification sequence, a labeled-binding-reagent binding sequence, a labeled-binding-reagent binding region, a molecular-marker binding sequence, a labeled-binding-reagent binding sequence, and a reverse amplification sequence. It is noted that some bivalent binding reagents are provided that bind to different sequences on the same molecular marker; as described herein above, such multiple bivalent binding reagents binding the same marker provide the weights that each molecular marker target contributes towards the basis measurement.

The foregoing bivalent binding sequences may be prepared by any method for preparing oligonucleotide sequences, and amplified using known methods. In one embodiment, PCR was used to add a T7 promotor sequence converting the ssDNA to dsDNA then performing an in-vitro transcription to convert the dsDNA to ssRNA. A reverse transcription is then used to convert the ssRNA to ssDNA. Each foregoing steps amplifying the total number of molecules. In another embodiment, asymmetrical PCR is used that produces an excess of ssDNA directly. In another embodiment, a rolling circle approach is used where the initial template is circularized then amplified into a long ssDNA strand consisting of many repeats of the template before being cleaved back to short template size ssDNA. These are merely non-limiting examples of methods that the oligonucleotide reagents disclosed here can be amplified to high quantities for the uses herein. In other embodiments, oligonucleotide reagents may be purified by any of many methods known in the art, such as but not limited to phenol chloroform extraction to remove proteins then a dialysis column to concentrate and buffer exchange oligonucleotides into small volumes of water. In other examples, alcohol precipitation protocols for concentrating oligonucleotide reagents as well as Speedvac where the solvent is evaporated to concentrate the reagents. Cleavage of the amplification regions may be achieved by restriction digestion, though this is not required for the oligonucleotides to carry out their intended purposes.

Oligonucleotide-based labeled binding reagents comprise an oligonucleotide sequence that binds to a labeled-binding-reagent binding sequence of a bivalent binding reagent, and a detectable label such as a fluorescent dye. In some embodiments the dye is covalent bound to the oligonucleotide region of the labeled binding reagent. In some embodiments, the dye is reversibly linked to the oligonucleotide region of the labeled binding reagent, for example using a disulfide bond, such that it can be cleaved (reduced) and removed for successive imaging using labeled binding reagents incorporating the same dye.

Non-limiting examples of labeled binding reagents are provided in Table 2 below. The table shows the sequence of the labeled binding reagent, and the sequence of the labeled-binding-reagent binding sequence(s) on the bivalent binding reagent to which it binds. Such labeled binding sequences are merely exemplary of those useful for the purposes disclosed herein; a skilled artisan will easily modify the design to accommodate other means for carrying out the teaching herein, including using non-oligonucleotide based reagents.

This disclosure encompasses the bivalent binding reagents and labeled binding reagents disclosed herein in Tables 1 and 2, including the bivalent binding reagents with and without either one or both of the amplification sequences, and the labeled binding reagents with and without a bound dye. As noted herein, the Cy5 dye used in the example (Cyanine5, Cy5 acid, CAS Registry No. 1032678-07-1, e.g., from BroadPharm) is representative of any of numerous dyes useful for the purposes herein, and the sample preparation and imaging protocol will guide the use of a single dye with successive imaging, the use of multiple different dyes and simultaneous imaging to scan those multiple dyes, or a different dye for each labeled binding reagent and simultaneous scanning for all dyes. Resources for other dyes useful for the purposes herein are found in: Beliveau et al., 2014, Visualizing genomes with Oligopaint FISH probes, Curr Protoc Mol Biol 2014 Jan. 6; 105:14.23.1-14.23-20. Non-limiting examples of other dyes useful for these purposes include Alexa.Fluor.350, Alexa.Fluor.405, Alexa.Fluor.488.H2O, Alexa.Fluor.532, Alexa.Fluor.610, Alexa.Fluor.633, ATTO.430LS, ATTO.490LS, ATTO.565, BD.Horizon.V450, BUV395 . . . BD.Horizon.Brilliant.Ultraviolet.395, BUV496 . . . BD.Horizon.Brilliant.Ultraviolet.496, BUV563 . . . BD.Horizon.Brilliant.Ultraviolet.563, BUV661 . . . BD.Horizon.Brilliant.Ultraviolet.661, BV421 . . . BD.Horizon.Brilliant.Violet.421, BV480 . . . BD.Horizon.Brilliant.Violet.480, BV510 . . . BD.Horizon.Brilliant.Violet.510, BV570 . . . BD.Horizon.Brilliant.Violet.570, BV605 . . . BD.Horizon.Brilliant.Violet.605, BV650 . . . BD.Horizon.Brilliant.Violet.650, CF405L, CF405M, CF405S, CF430, Cy3, Cy3.5, Cy5, DY.350XL, DY.360XL, DY.375XL, DY.380XL, DY.395XL, DY.480XL, DY.485XL, DY.510XL, DY.521XL, NovaBlue.530, NovaBlue.610, NovaBlue.660, NovaYellow.570, NovaYellow.660, POPO.1, PromoFluor.350LSS, PromoFluor.370LSS, PromoFluor.375LSS, PromoFluor.488LSS, PromoFluor.500LSS, PromoFluor.510LSS, PromoFluor.520LSS, PromoFluor.532, Super.Bright.436, Super.Bright.600, Super.Bright.645, Super.Bright.702, and SYTO.40, Other dyes include BODIPY 630/650-X, LC Red 640, BODIPY 650/665-X, Alexa Fluor 647, Alexa Fluor 660, Cyanine5.5, Alexa Fluor 680, Alexa Fluor 700 and Alexa Fluor 750. The disclosure is not so limited by such selections and may depend on the availability of instruments to perform the requisite scanning to achieve the purposes herein, the time necessary for the scan and need for the resultant data, and other factors which do not deviate from the intended purposes disclosed herein. For hyperspectral scanning, a selection of dyes includes DY.360XL, CF405S, NovaBlue.530, NovaYellow.570, Alexa.Fluor.633, DY.375XL, ATTO.490LS, BUV395 . . . BD.Horizon.Brilliant.Ultraviolet.395, BUV496 . . . BD.Horizon.Brilliant.Ultraviolet.496, BUV563 . . . BD.Horizon.Brilliant.Ultraviolet.563, BUV661 . . . BD.Horizon.Brilliant.Ultraviolet.661, POPO.1, DY.380XL, BV570 . . . BD.Horizon.Brilliant.Violet.570, BV605 . . . BD.Horizon.Brilliant.Violet.605, Super.Bright.645, CF430, PromoFluor.350LSS, DY.395XL, PromoFluor.520LSS, DY.485XL; NovaYellow.660, PromoFluor.500LSS, PromoFluor.510LSS, and Super.Bright.702.

Other designs of the labeled reagent set will be dictated by the encoding method used. Labeled binding reagents are prepared using dye-oligonucleotide or dye-protein (antibody or antigen-binding fragment) chemistries well known in the art. In other embodiments, other types of tags are labeled using appropriate chemistries, such as but not limited to bispecific antibodies (a bivalent reagent recognizing for example a protein target and a detectably labeled antigen).

In other embodiments, such further layers of oligonucleotide reagents are provided that relate encoding to a taxonomic tree of cell types. Such modification of the procedure as described would be correspondingly applied to the other steps in the method. In other embodiments, such further one or more layers may be achieved with antibodies or antigen-binding fragments thereof, and corresponding antigens comprising such bi-functional reagents. In other embodiments, similar methods are applied to labels on different types of tags.

In one embodiment, the dyes used in the preparation of the binding reagents are imaged using hyperspectral imaging, wherein all dyes at a particular location within the specimen are imaged simultaneously and the quantitative information on each dye present at that location recorded. In an alternative embodiment, the dyes are imaged using a sequential wavelength-limited imaging, the sample washed, and reimaged using stepwise imaging methodology. In other words, in one non-limiting example, a technician may stain the sample using Cy3-labeled binding reagents and then image in the Cy3 emission wavelength; wash the sample; then re-stain using Texas Red-labeled binding reagents and then image in the Texas Red emission wavelength; wash again; and so on. The various captured wavelengths may then be layered into a composite image. Alternate embodiments and methods for imaging are described in the paragraph on step (f), below.

Incubating the Specimen with the Bivalent Binding Reagents and the Labeled Binding Reagents (Step e)

Staining with the two types of binding reagents as specified above may be performed on specimens that are prepared for the subsequent imaging step. For thin sections wherein two-dimensional imaging is sufficient, standard specimen staining protocols may be utilized. For thicker specimens where three-dimensional information is needed, tissue preparation is typically required such as involving tissue clearing. In one non-limiting embodiment, the specimen is embedded in a hydrogel matrix. In one non-limiting embodiment, the labeled binding reagents comprise a moiety that will allow coupling to the hydrogel, such as acryloyl-containing moieties that can be polymerized into an acrylamide gel (see, e.g., Moffitt et al., PNAS Dec. 13, 2016, 113 (50) 14456-14461). In some embodiments, the specimen after polymerization into the gel may be isotropically expanded to facilitate hyperspectral or any other type of imaging. In some embodiments, after the bivalent binding reagents and labeled binding reagents have localized to their binding sites, the specimen permeated with polymerizable monomers and the labeled binding reagents cross-linked into the hydrogel matrix, the specimen may be cleared of the specimen, leaving the labeled binding reagents in the positions of the original cells they were designed to locate.

Because the bivalent binding reagents are typically larger than the labeled binding reagents, in particular for nucleic acid based reagents (e.g., about 150 nucleotides vs. about 20 nucleotides), incubation with the former requires a longer period than the latter. In some embodiments, the molecular-marker binding region of the bivalent binding reagent needs to bind RNA targets that are fixed with potentially some secondary structures. Therefore, when oligonucleotide-based reagents are used, the hybridization of bivalent binding reagents to RNA takes a long time (e.g. 12 hours) but has to occur only once prior to imaging. The second hybridization type is using labeled binding reagents that hybridize to the bivalent binding reagents. In some embodiments, this step is very fast (about <15 minutes) due to the simplicity of binding to bivalent binding reagents and the short length of labeled binding reagents (about 20 base pairs+fluorophore). In some embodiments, multiple rounds of incubation of the sample with labeled binding probes is needed, e.g., 8 times assuming 3 color imaging of 24 readout rounds. Optimization of the incubation periods to improve the results and/or reduce the incubation times of the various reagents is embraced herein.

In some embodiments, the incubating of the specimen with the bivalent binding reagents is performed before the incubating with the labeled binding reagents. In some embodiments, the incubating with the bivalent binding reagents is longer than incubating with the labeled binding reagents. In some embodiments, the incubating with the bivalent binding reagents is performed at the same time as with the labeled binding reagents. In some embodiment the specimen is washed after incubating with the bivalent binding reagents and before the incubating with the labeled binding reagents. In some embodiments the labeled binding reagents are added after the bivalent binding reagents. In some embodiments the specimen is washed after incubation with the labeled binding reagents.

In some embodiments, the specimen is prepared to maximize penetration of bivalent binding reagents. In some embodiments, electrophoretic fields are used to uniformly stain the specimen. In some embodiments, stochastic electrotransport is used, wherein the directionality of the electric field is randomly changed over time to actively disperse molecules to uniformly stain thick gels.

Imaging Positions within the Specimen (Step f)

In one embodiment, for hyperspectral imaging in two dimensions, in one embodiment a hyperspectral epi-fluorescence/confocal microscope can be used. In some embodiments, the hyperspectral light-sheet microscope may use high-transmittance tunable filters that change the position of the bandwidth as a function of the angle of the filter. In some embodiments, the hyperspectral light-sheet microscope comprises a moving stage and laser strobing.

For three-dimensional samples, in one embodiment, a hyperspectral light-sheet microscope may be used. Non-limiting examples include that described by Jahr et al., NATURE COMMUNICATIONS 2015; 6:7990; Lavagnino et al., BIOPHYSICAL J 2016; 111:409-417; Xu et al., OPTICS EXPRESS 2017; 25(25):31159-31173).

In other embodiments, imaging may be achieved using standard fluorescence imaging, light sheet imaging, or flow cytometry. In some embodiments, step (f) is accomplished by non-optical sensing methods such as mass spectrometry. The method disclosed herein is not limited in any way to the particular method of detecting the labeled tags in the specimen, and the skilled artisan will be easily guided to the appropriate method by considering the number of labels to be measured, the time available for the assessment (e.g., acutely deciding a patient's course of therapy from a biopsy assessed using these methods), the thickness of the specimen, the available equipment where the method is carried out, and other considerations that are fully embraced herein.

In one embodiment, the imaging at positions throughout the specimen to detect the labeled binding reagents and extent of labeling is obtained sequentially or simultaneously. In some embodiments, the specimen is imaged for all dyes at each location in the specimen simultaneously. In some embodiments, the specimen is imaged for each dye sequentially at each location in the specimen. In some embodiments, the specimen is incubated with all of the bivalent binding reagents and the subsequent incubating with the labeled binding reagents may be simultaneous or sequential, with, in some embodiments, imaging after each sequential incubation with each labeled binding reagent or a subset of the labeled binding reagents. Thus, in some embodiments, the imaging is performed batchwise to detect one or more dyes each scan. In some embodiments, after such partial imaging of the sample, the one or more labeled binding reagents are washed out of the specimen before the next one or more labeled binding reagents are incubated then imaged. In some embodiments, the washing out comprises removing the labeled binding reagents. In other embodiments, the dyes of the labeled binding reagents are quenched or otherwise made to not interfere with subsequent imaging of the same or different dyes.

Correlating Staining with Cell Type (Step g)

To subsequently decode the captured image data as described above and assign cell types, the following steps are carried out:

If the recursive partitioning method in step c was used:
(1) Start at the root and determine if the total number of fluorophore molecules of level one that was measured for that cell is below or above the threshold learned when the logistic regression was fit. Assign 0/1 to that cell based on the stain and threshold.
(2) Recursively traverse the classification tree and at each branch repeat the determination described for the root, i.e., is the number of measured fluorophore molecules is below/above the learned threshold.
(3) The result of the recursive procedure is a set of 1/0 that fully determines the cell type of a cell as they specify exactly where that cell is in the cell type binary classification.

If the dPNMF method in step c was used:
(1) After measurements, use the values per cell and based on the fitted Naive Bayes classifier determine the most probable cell type given the aggregate staining measurements. The number of fluorophores, i.e., the dimensionality of the learned weight matrix in the dPNMF, is user-defined.

Empirically, a test of this method, as shown below in Example 4, achieved a performance of dPNMF with 24 dimensions. In other words, the abundance of ˜9,000 markers (e.g., RNA types) was mapped into 24 aggregate measurements such that the information on the label in each of these measurements is preserved.

In other embodiments, if a different method for establish the relationship (encoding) was used to create the set of bivalent binding reagents and labeled binding reagents for the particular type of specimen, in a similar fashion the data from the imaging of the specimen may be decoded following that encoding process.

### Identify Locations of Specific Cell Types (Step h)

The data on specific cell types and their locations obtained in step (g) are provided as a map or other data format to identify cell type locations within the specimen.

In one embodiment, the molecular markers are nucleic acid polymers. In a preferred embodiment, the nucleic acid polymers are RNA. In an alternative embodiment, the molecular markers are protein, which may be any of a secreted protein, cell-surface protein, receptor, transcription factor, antibody, or a combination thereof. In other embodiments, the molecular markers are metabolites, lipids, carbohydrates including polysaccharides, glycolipids, vitamins, fatty acids, co-factors, pigments, metals, or any other biochemicals or compounds, organic or inorganic, found within a biological system, or any combination thereof. In some embodiments, the methods disclosed herein are applied to two or more types of markers (e.g., proteins and nucleic acids) using the appropriate reagents for each type of marker, which may be performed concurrently (i.e., incubation with bivalent binding reagents for the proteins and nucleic acids expressed by cells in the specimen; incubation with labeled binding reagents that bind to the respective protein or nucleic acid binding bivalent binding reagents).

Tags (i.e., the molecular-marker binding portion of the bivalent binding reagents) useful for their detection in accordance with the teaching herein include but are not limited to antibodies and antigen-binding fragments thereof, ligands, lectins, receptors, chelators, etc.

In one embodiment, steps (a), (b) and (c) are performed for a particular type of biological specimen wherein the specimen comprises a plurality of known cell types (e.g., known from the literature) and among the known cell types within the specimen from which the plurality are selected for locating, in step (a), the known molecular markers of each cell type is obtained from the literature, for step (b). Based upon the selection of molecular markers in step (b), step (c) may be carried out to identify the markers to be detected, and step (d) the design of the reagents. Thus, steps (a)-(d) are carried out for each particular type of specimen and cells therein of interest in locating. In one embodiment, the remainder of the steps are carried out on the specimen.

In one embodiment, steps (a), (b) and (c) are carried out in silico.

In another embodiment, step (b) may further comprise organizing the specific cell types into a hierarchical taxonomy according to the plurality of known molecular markers. In some embodiments, the number of different detectable labels of step (d) may equal to the number of hierarchical levels of the hierarchical taxonomy.

In yet another embodiment, step (c) is accomplished using a dimensionality reduction process. In still another embodiment, step (c) is accomplished using recursive partitioning. In another embodiment, step (c) is accomplished using machine learning to design the encoding. In an alternative embodiment, step (c) is accomplished using discriminant projection non-negative matrix factorization (dPNMF). In an aspect, dPNMF comprises the steps of (i) fitting a dPNMF model to training data; (ii) fitting a classifier to one class per cell type; and (iii) creating a staining profile for each cell type according to a weighting, whereby the number of cell labels per molecular marker approximates weighting. In an aspect, the classifier of step (ii) is a Naïve Bayesian classifier. In another aspect, the classifier of step (ii) is KNN.

In one embodiment, step (d) is accomplished using direction from step (c) as to the preparation of the set of labeled binding reagents. In one embodiment, the labels are dyes that are individually detectable in a single location within the specimen using hyperspectral imaging.

In one embodiment, step (f) is accomplished by hyperspectral scanning of the specimen. In one embodiment, hyperspectral epifluorescence/confocal microscopy is used. In one embodiment, hyperspectral light-sheet microscopy is used. In other embodiments, step (f) is accomplished by standard fluorescence imaging, light sheet imaging, or flow cytometry. In some embodiments, step (f) is accomplished by non-optical methods such as mass spectrometry.

In some embodiments, the specimen is incubated with all of the bivalent binding reagents, but the incubating with the labeled binding reagents may be simultaneous or sequential, with, in some embodiments, imaging after each sequential incubation with each labeled binding reagent.

In one embodiment, the data obtained from step (f) is converted to locations of particular cell types within the specimen using the correlating of step (g), which is based upon the relationships established in step (c). In one embodiment, step (c) is an encoding step, and step (g) is a decoding step.

In one embodiment, the locations of the cell types within the specimen in step (h) are used diagnostically to identify, for example, a disease state or the potential for a diseases state to develop based upon the locations of particular cell types within the specimen.

These and other aspects of the embodiments described herein will be provided in the ensuing descriptions of the drawings and detailed description of the embodiments.

The following numbered embodiments are embraced by the disclosure herein.
1. A method for identifying the specific locations of a plurality of specific cell types within a population of cells in a biological specimen, the method comprising the steps of:
   a. selecting the plurality of specific cell types within the specimen, based on the origin of the specimen and the known cell types anticipated to be present therein;
   b. determining among those specific cell types the known extent of presence or absence of a plurality of known molecular markers of each specific cell type therein;
   c. establishing a relationship using a subset of the plurality of molecular markers among all specific cell types therein, wherein the extent of expression of the subset of molecular markers or lack thereof by each of the specific cell types can maximally differentiate each specific cell type from each other specific cell type;
   d. preparing a set of labeled binding reagents that detectably label each of the subset of molecular markers, wherein each binding reagent is detectably labeled from a finite selection of a plurality of types of individually and simultaneously detectable labels, wherein each binding reagent is labeled with one type of detectable label and number of such labels per binding reagent, such that the set of labeled binding reagents, when bound to the subset of molecular markers expressed by each specific cell type in the sample, provides an extent of labeling that maximally differentiates each specific cell type from each other specific cell type;
   e. staining the specimen with the labeled binding reagents;
   f. imaging positions throughout the specimen to detect the labeled binding reagents and extent of labeling thereof, wherein the detection of the labeled binding reagents at each position is obtained simultaneously, to provide the positions of the subset of molecular markers and extent of expression thereof;
   g. correlating the extent of expression of the subset of molecular markers at positions throughout the specimen using the dimensionality reduction analysis based on the pre-established relationship between the extent of expression of each of the plurality of molecular markers or lack thereof by each of the specific cell types that differentiates each specific cell type from each other specific cell type, to establish a highest-probability estimate of the presence of a specific cell type at a specific location within the specimen; and
   h. identifying the specific locations of the specific estimated cell types within the specimen.
2. The method of embodiment 1 wherein the known molecular markers are nucleic acid polymers.
3. The method of embodiment 2 wherein the nucleic acid polymers comprise RNA.
4. The method of embodiment 1 wherein the known molecular markers are peptides, whole proteins, and/or protein fragments.
5. The method of embodiment 4 wherein the proteins are any of a peptide, nuclear protein, cytosolic protein, mitochondrial protein, secreted protein, cell-surface protein, receptor, transcription factor, antibody, or any combination thereof.
6. The method of embodiment 1 wherein step (b) is accomplished using scRNAseq.
7. The method of embodiment 1 wherein step (c) is accomplished using a dimensionality reduction process.
8. The method of embodiment 1 wherein step (b) further includes organizing the specific cell types into a hierarchical taxonomy according to the plurality of known molecular markers.
9. The method of embodiment 7 wherein step (c) is accomplished using recursive partitioning.
10. The method of embodiment 7 wherein the number of detectable labels of step (d) is equal to the number of hierarchical levels of the hierarchical taxonomy of step (b).
11. The method of embodiment 1 wherein step (c) is accomplished using discernment projection non-negative matrix factorization (dPNMF).
12. The method of embodiment 11 wherein dPNMF comprises
   a. fitting a dPNMF model to training data;
   b. fitting a classifier to one class per cell type; and
   c. creating a staining profile for each cell type according to a weighting, whereby the number of cell labels per molecular marker approximates weighting.
13. The method of embodiment 12 wherein the classifier is a Naïve Bayesian classifier.
14. An apparatus configured to implement any of the methods of embodiments 1-13.

### EXAMPLES

The following examples are put forth so as to provide persons having ordinary skill in the art with a complete disclosure and description of how to make and use the subject disclosure, and are not intended to limit the scope of disclosure. Efforts have been made to ensure accuracy with respect to the numbers used (e.g., quantities, amounts, temperature, concentrations, etc.) but some experimental errors and deviations should be allowed for. Unless otherwise indicated, parts are parts by weight, molecular weight is average molecular weight, temperature is in degrees Celsius; and pressure is at or near atmospheric pressure. Overall, encoding and decoding (e.g., creating the bivalent binding reagents and labeled binding reagents, and analyzing the images created therefrom) is a statistical learning task, and like many statistically learning tasks there are several variants one could implement. This disclosure is no limited to any particular encoding and decoding methods or algorithm. In each example a reference dataset is used with many exemplary cells having the following properties: (1) abundance of markers of interest (e.g., protein or RNA); and (2) cell type label. This as conceptualized as a matrix wherein the first n columns are abundance values and the n+1 column is a category, i.e., the cells type of the specific cell.

### Example 1: dPNMF Dimension Reduction Data

Preliminary data from mouse whole coronal thin sections show that dredFISH measurements contain detailed information on cells' transcriptional states. These data combined with the lower magnification optics and the very bright signal generated from summing expression of thousands of genes, make dredFISH an ideal approach for cell-type mapping of large volumes. This example shows the feasibility of directly measuring multiple weighted sums of gene expression, an informative low dimensional approximate representation of gene expression, without measuring individual gene expression levels.

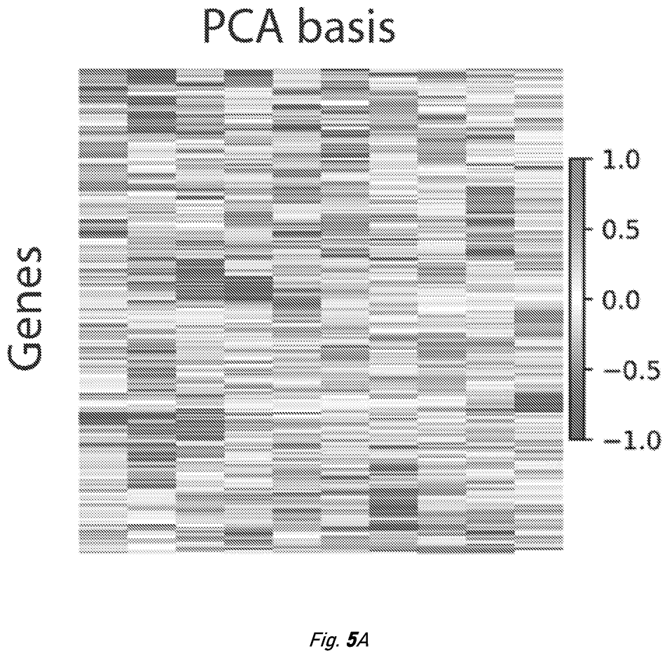

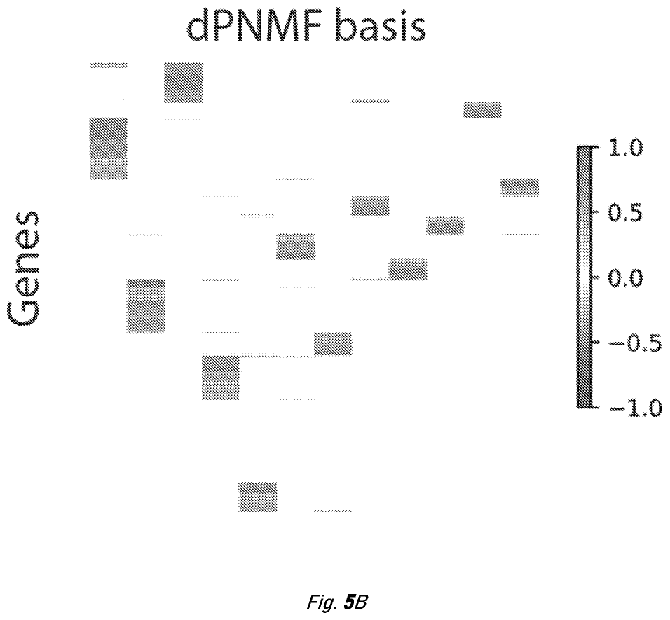

To validate these premises, we first designed the projection matrix of gene expression to lower dimensional space (DPNMF basis, FIG. 5B). The design example is based on 24 dimensions and was created using a cell type balanced scRNAseq dataset collected by Brain Initiative Cell Census Network (BICCN). Based on this design, we created a DNA oligo pool with more than 92,000 oligos targeting >9000 genes. Weights in the DPNMF matrix were rescaled from 0 to 100 and rounded to the closest integer. As shown in FIGS. 4A-4C, the number of different oligos that map a given gene to a specific basis is simply the weight in the scaled and rounded DPNMF matrix. DNA oligos were synthesized following established protocols that were first developed for OligoPaint DNA FISH (Beliveau et al., 2014, Visualizing genomes with Oligopaint FISH probes, Curr Protoc Mol Biol 2014 Jan. 6; 105:14.23.1-14.23-20). Oligonucleotide sequences of exemplary bivalent binding reagents and oligonucleotide portions of the labeled binding reagents among those used in this study are described in Example 6, Tables 1 and 2.

We next stained and imaged a mouse coronal section (FIG. 6). The section was stained using bivalent binding reagents based on the DPNMF projection matrix. Using multiple rounds of imaging, the sample was imaged 24 times using Cy5 labeled binding reagents. The labeled binding probes are identical to those used in MERFISH and include disulfide bonds between the fluorophore and the oligo (see, for example, Zimmermann et al., Thiol based, site-specific and covalent immobilization of biomolecules for single-molecule experiments, 2010, Nat Protoc 5(6):975-985. Moffitt et al., 2016, High-performance multiplexed fluorescence in situ hybridization in culture and tissue with matrix imprinting and clearing, PNAS 113(50):14456-14461). Oligonucleotides bound to dyes by a reducible disulfide bond may be obtained, for example, from IDT Custom DNA Oligos. The Cy5 fluorophore was removed between rounds of imaging using simple tris(2-carboxyethyl)phosphine (TCEP) reduction and fluidic washing. The choice of 24 dredFISH dimensions balances degree of approximation and acquisition time. Further optimization of the number of dimensions is embraced herein. FIG. 6 shows 12 of the 24 dredFISH measurements. These panels show the raw dredFISH measurements after a simple z-score scaling, i.e. subtraction of sample mean and division by sample standard deviation. The clear spatial patterns indicate that dredFISH approximation contains abundant data on the spatial distribution of gene expression programs.

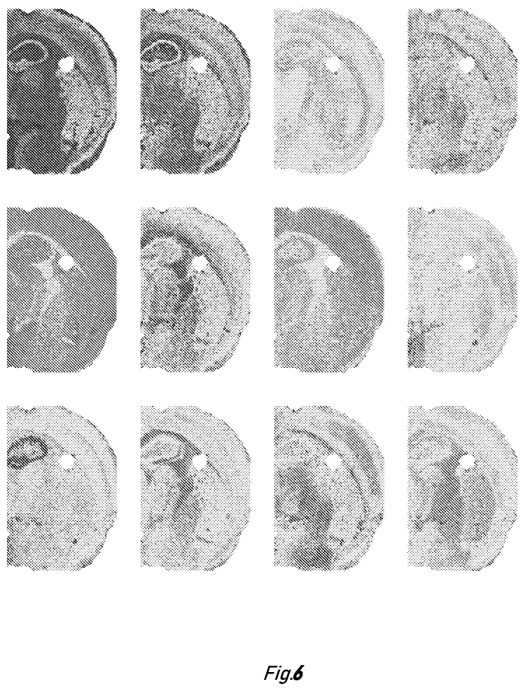

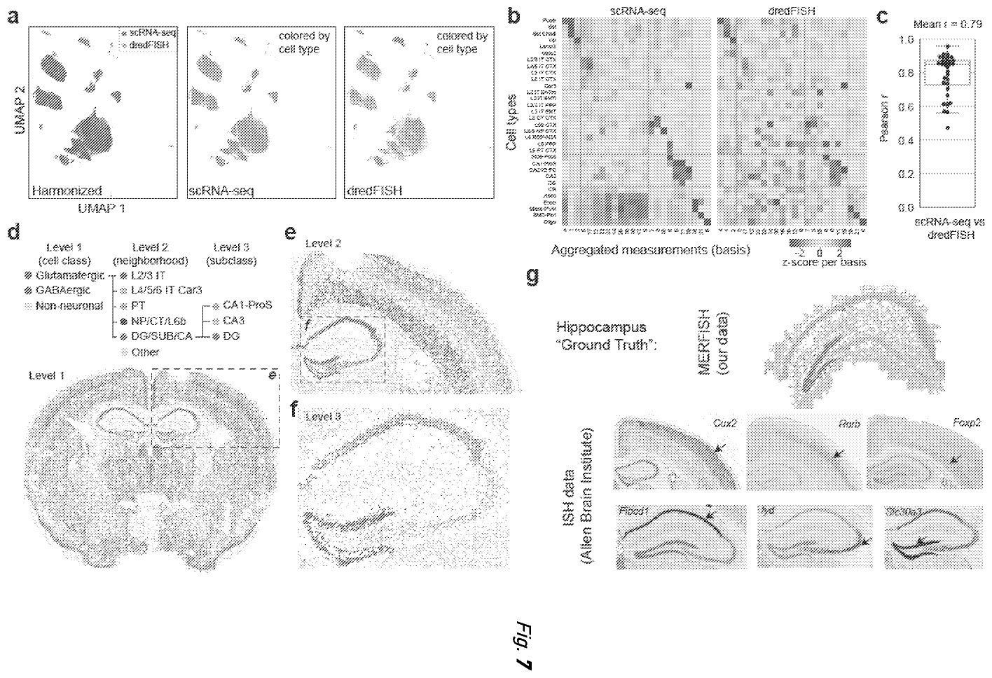

We further validated that the observed spatial patterns provide enough information for integrating dredFISH and scRNAseq (FIG. 7). The first step in this integration approach is to harmonize dredFISH data to scRNAseq reference. Harmonization is needed for two reasons: (1) The scale of dredFISH measured on the microscope is different from the counts provided by scRNAseq; and (2) The cell type composition of scRNAseq does not reflect the composition in the organ. Our harmonization approach uses an expectation-maximization (EM) algorithm in which we maximize classification accuracy and at each step infer cell type composition. At each step, we resample the reference given the current composition estimate. We take advantage that the cell types in the brain are labeled to types at multiple levels. Level-1 is coarse and only has three types (excitatory neurons, inhibitory neurons, and non-neuron cells). Level-2 has 8 cell types and level-3 44 distinct cell types. Harmonization is achieved by repeating z-score and cell type classification at all the predefined levels. We first z-score the entire data and classify cells to level-1 types. Then, the subset of cells in each of the level 1 types is z-scored again and classified into the subtypes of level-2. This is repeated again for level 3. Overall, this harmonization algorithm brings the two distinct datasets into the same space where they can be directly compared (FIG. 7A). After harmonization, we perform label transfer using kNN classification. FIG. 7B shows the average basis representation of detected cell types calculated based on scRNAseq and dredFISH. The overall agreement between these two techniques is high (mean Pearson correlation 0.79, FIG. 7C). The cell type labels we inferred (FIG. 7 D-F) match the known ground-truth data from two different sources (FIG. 7G). The first is the classification of cells into distinct types in the hippocampus based on our own MERFISH data and the second is expression patterns of key marker genes from the Allen ISH atlas. Overall, the qualitative agreement between our cell type inference and the ground truth provide strong support for the method: dredFISH measurements of cellular transcriptional states integrated with scRNAseq produces accurate supervised cell types inference.

Thus, the identified clusters spatially match the anatomy of the mouse brain including the six layers in the cortex, different components of the hippocampus, hypothalamic nuclei, and thalamic nuclei. To further dissect the information content in dredFISH dimensions we chose a cluster that represented neurons in the hippocampus and repeated the unsupervised clustering only for these cells. Spatial mapping of the identified subclusters within hippocampus neurons matches the known spatial position of CA1, CA3, and DG neurons. The ability of a standard unsupervised clustering algorithm to identify clusters that match known neuronal types validates the methods disclosed herein for directly measuring an abstract low-dimensional representation of gene expression without measuring individual gene expression levels.

### Example 2: Further Analysis of Imaging and Cell-Type Classification Data

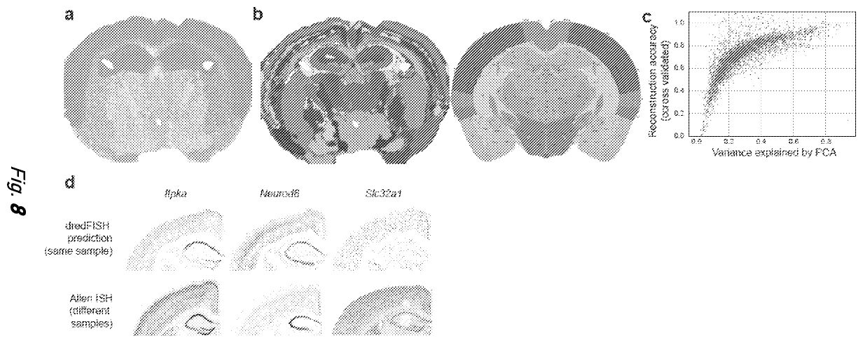

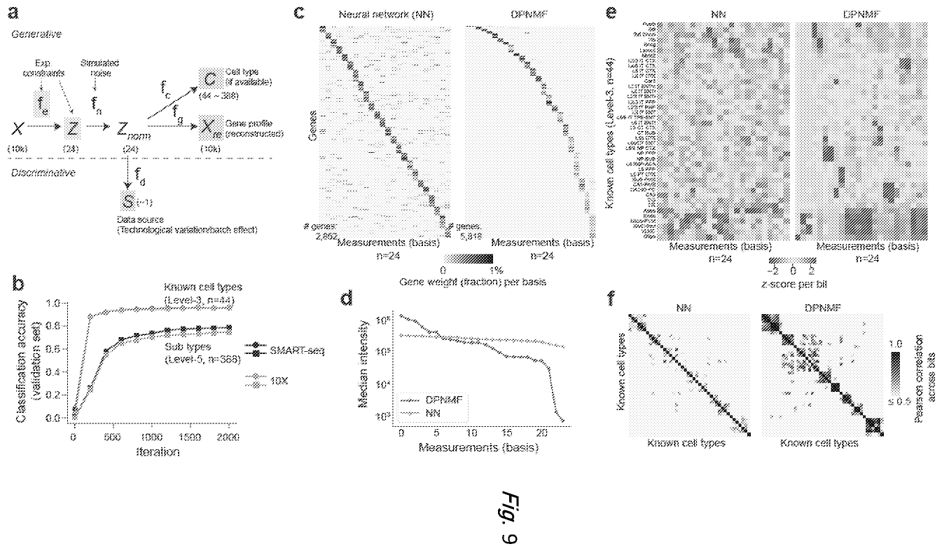

We further analyzed the information contained in dredFISH measurements for data analysis tasks common in spatial transcriptomics analysis: unsupervised classification into types, cell-cell interactions, regions identification, and gene expression reconstruction (FIG. 8). Currently, the Brain Initiative only defines cell types for the isocortex and hippocampus formation (FIG. 8A grayed out region). While the DPNMF projection matrix we designed was not optimized to classify the 69,683 cells outside the reference regions, it still contains relevant information that could be used for unsupervised clustering. Using standard Leiden graph based clustering we classified cells into clusters using their dredFISH approximate transcriptional states (FIG. 8A). Combining the supervised cell types calls (FIG. 9D-F) and the unsupervised classes (FIG. 8A) we noticed very distinct local abundances of subsets of cell types. Using a topic modeling approach we performed a Latent Dirichlet Allocation (LDA) analysis using local cell composition. FIG. 8B shows the identified anatomical regions in comparison to known anatomical regions in the brain. We next tested whether we could decode individual gene expression levels using the approximate dredFISH measurements. As dredFISH relies on patterns of gene expression, we expect gene expression reconstruction accuracy to change depending on the pattern of expression of each gene. To assess this relationship we compared reconstruction accuracy using reference data and kNN regression to reconstruction based on PCA using all components that had more signal than permuted data (81 components). As expected, accuracy of gene reconstruction using kNN regression is high for genes whose expression matches broad expression patterns (FIG. 8C) and low in cases where PCA reconstruction accuracy is low. A few examples of gene expression reconstructions are shown in FIG. 8D. Overall, these analyses demonstrate that dredFISH approximate measurements of cellular transcriptional state allows data analysis tasks common in spatial transcriptomics.

Collectively, the data presented here demonstrate how the dredFISH principle works in practice and shows overall high cell type classification accuracy.

### Example 3: Recursive Partitioning Encoding Method

All cell types are organized into a binary classification tree. The tree can be learned directly from observations determining among those specific cell types the known extent of expression or lack thereof of a plurality of known molecular markers of each specific cell type, using hierarchical clustering or using prior knowledge.

Provided with this cell-type tree (i.e., cell taxonomy), the following operations are performed:
(1) Assign 1/0 labels to all cells based on where their cell type is in relation to the initial split. For example, all cells with types that are on the left side of the split will get “1” all the cells that have type on the right will get “0”. In the brain example above, the first split is to “non-neurons” and “neurons” and all cells will be labeled accordingly.
(2) Fit a regularized logistic regression where the dependent variable is the “1”/“0” we assigned above, and the independent variables are the abundances of all markers. The results of the fit are the coefficients and threshold. The coefficients are the weights for each marker such that when all the abundance of each marker are multiplied by these coefficients we obtain maximal separation between the “0” and “1” class. The threshold is a value where above it the product of the coefficients times the abundance should be mapped to “1” and below it to “0”. The regularization is there to enforce that all the weights are non-negative.
(3) Recursively traverse the tree, and at each step perform an analysis task similar to what was done for the root. Label all cells that are below the left side of the branch as “1” and the others as “0”, fit a logistic regression and obtain the same coefficients and threshold for each of the splits in the tree. Again, use regularization to ensure that all coefficients are non-negative.
(4) Use the weight coefficients to create an encoding. The number of fluorophores we need is the depth of the cell type tree. For each of these fluorophores, we design a staining such that the number of fluorophore molecules that bind that marker is the value of the coefficient learned through the logistic regression.

Provided with these encoded machine-learned data, we will create a staining protocol wherein the number of unique fluorophores equals the delph of the taxonomic tree, i.e., a vector of values. To later decode that vector and assign cell types, we do the following:
(1) Start at the root and determine if the total number of fluorophore molecules of level one that we measured for that cell is below or above the threshold we learned when the logistic regression was fit. Assign 0/1 to that cell based on the stain and threshold.
(2) Recursively traverse the classification tree and at each branch repeat the determination described for the root, i.e., is the number of measured fluorophore molecules is below/above the learned threshold.
(3) The result of the recursive procedure is a set of 1/0 that fully determines the cell type of a cell as they specify exactly where that cell is in the cell type binary classification.

### Example 4: Discernment Projection Non-Negative Matrix Factorization (dPNMF) Encoding Method

Discernment projection non-negative matrix factorization (dPNMF), such as the method described in Guan et al., PLoS ONE, 2013 Dec. 20; 8(12):e83291, provides a means for learning from the data a dimensionality-reduced representation that can be obtained using only non-negative weights.

The encoding step establishing the relationship between molecular markets and cell types, is achieved by dPNMF as follows:
(1) Fit dPNMF model to the training data. The results of the fit is a weight matrix that maps between the markers to a dimensionality-reduced space in a way that maximizes the information in-that space about original marker abundance and cell type label.
(2) In the dimensionality-reduced space, fit a multi-class Naïve Bayesian classifier with one class per cell type. Many other classifiers could be used here and this step will only occur in silico during decoding.
(3) Implement the weights by creating staining where the number of fluorophore molecules per marker approximate the learned non-negative weight.
(4) After measurements, use the values per cell and based on the fitted Naive Bayes classifier determine the most probable cell type given the aggregate staining measurements.

The number of fluorophores, i.e., the dimensionality of the learned weight matrix in the dPNMF, is user-defined.

Empirically, a test of this method achieved a performance of dPNMF with 24 dimensions. In other words, we mapped the abundance of ˜9,000 markers (e.g., RNA types) into 24 aggregate measurements such that the information on the label in each of these measurements is preserved.

### Example 5. Neural Network Probe Design

As shown in the preceding examples, DPNMF was found to provide reliable encoding. DPNMF provides an excellent projection, as can be gleaned from the data shown above. However, other neural network-based optimization schemes provide alternate methods for achieving the objectives of the methods described herein. In one embodiment, the lack of uniformity across rounds using DPNMF may reduce the overall information contained in the encoding scheme. For example, there are more than 1000-fold differences between the dimmest to the brightest basis (FIG. 9D). This difference mostly arises from differential expression of genes and not from the number of probes we designed. The 1000-fold difference creates a technical difficulty as the measurements need to be sensitive enough to capture small differences in the dimmer readout rounds without saturating the bright rounds.

The DPNMF projection is replaced with a neural network-based optimization (model shown in FIG. 9A): the new algorithm uses a multilayer neural network. The first layer encodes the mapping between genes and readout rounds and is subjected to experimental constraints (fe). The sparsity, non-negativity, intensity, and uniformity of the matrix, are achieved by adding regularization terms during training. To increase the robustness of the design, we added Poisson noise (fn) and an adversarial network (fd) that discriminates between different types of scRNAseq data. This process ensures that the designed projection is isolated from technical challenges in scRNAseq data, such as batch effects. Finally, the last layer uses the latent state to classify cells into known types and reconstruct gene expression (decoder fg). The new design addresses the issue of dynamic range as can be seen in (FIG. 9D). The cell type signatures resulting from the new design show less self-similarity across cell types compared to the DPNMF design as can be seen in the cell type Pearson correlation matrices of both projections (FIG. 9F) and provide high classification accuracy using independent validation with scRNAseq data (FIG. 9B). The design shown above by testing different hyper-parameter values for the regularization constraints and overall network structure. For example, a systematic test of the effect of the dimensionality of dredFISH basis on classification accuracy will determine the required number of imaging rounds, and convert the top three designs created using the multilayer neural network optimization approach to create DNA oligo pools and are tested experimentally using mouse brain coronal sections and the MERFISH internal ground truth, as described above. These studies provide an improved alternative to the methods described above.

### Example 6. Reagent Sets for dredFISH

Among the examples described herein above, examples from among the more than 92,000 oligos targeting more than 9,000 genes are provided below, used in the methods disclosed herein for preparing the images shown in FIG. 6.

Table 1 lists 24 representative bivalent binding reagents (and Table 2, labeled binding reagents that bind them) used in the examples herein. The following oligonucleotides were prepared using PCR to add a T7 promotor sequence converting the ssDNA to dsDNA then an in-vitro transcription was performed to convert the dsDNA to ssRNA. A reverse transcription was then used to convert the ssRNA to ssDNA, each step amplifying the total number of molecules.

TABLE 1
Examples of dredFISH bivalent binding reagents for brain cell type scanning

| | Reagent | | Labeled- | Reagent | |
|---|---|---|---|---|---|
| | amplification | Molecular- | binding- | amplification | |
| | reagent 1 | marker | reagent | reagent 2 | |
| | (forward) | binding | binding | (reverse) | |
| Target | binding region | region | region | binding region | Entire bivalent binding |
| RNA | sequence | sequence | sequence | sequence | reagent sequence |
| Atp6v1h | TGGCCGTCG | GGTCTTC | AGAGTG | GCAGAATTT | TGGCCGTCGATTCCGT |
| | ATTCCGTGA | ATGGTGC | AGTAGT | CCTGGTGCG | GAATAGAGTGAGTAGT |
| | AT (SEQ ID | ATGTGGT | AGTGGA | GG (SEQ ID | AGTGGAGTAAGAGTGA |
| | NO: 49) | TCATCACC | GT (SEQ | NO: 50) | GTAGTAGTGGAGTAGG |
| | | (SEQ ID | ID NO: 1) | | TCTTCATGGTGCATGT |
| | | NO: 51) | | | GGTTCATCACCAAAGA |
| | | | | | GTGAGTAGTAGTGGAG |
| | | | | | TGCAGAATTTCCTGGT |
| | | | | | GCGGG (SEQ ID NO: 25) |
| Jph1 | TGGCCGTCG | GAGGCTT | TGTGAT | GCAGAATTT | TGGCCGTCGATTCCGT |
| | ATTCCGTGA | TTCTAGG | GGAAGT | CCTGGTGCG | GAATTGTGATGGAAGT |
| | AT (SEQ ID | ACCTTTTC | TAGAGG | GG (SEQ ID | TAGAGGGTATGTGATG |
| | NO: 49) | TTCTGGA | GT (SEQ | NO: 50) | GAAGTTAGAGGGTAGA |
| | | (SEQ ID | ID NO: 2) | | GGCTTTTCTAGGACCT |
| | | NO: 52) | | | TTTCTTCTGGATATGT |
| | | | | | GATGGAAGTTAGAGGG |
| | | | | | TGCAGAATTTCCTGGT |
| | | | | | GCGGG (SEQ ID NO: 26) |
| Gm1992 | TGGCCGTCG | AACAGAT | TGAAAG | GCAGAATTT | TGGCCGTCGATTCCGT |
| | ATTCCGTGA | CTCACTG | GAATGG | CCTGGTGCG | GAATTGAAAGGAATGG |
| | AT (SEQ ID | CACACGG | GTTGTG | GG (SEQ ID | GTTGTGGTAAACAGAT |
| | NO: 49) | GCATATCT | GT (SEQ | NO: 50) | CTCACTGCACACGGGC |
| | | (SEQ ID | ID NO: 3) | | ATATCTCATGAAAGGA |
| | | NO: 53) | | | ATGGGTTGTGGTATGA |
| | | | | | AAGGAATGGGTTGTGG |
| | | | | | TGCAGAATTTCCTGGT |
| | | | | | GCGGG (SEQ ID NO: 27) |
| Gm26901 | TGGCCGTCG | TGGAAGG | GGGTTG | GCAGAATTT | TGGCCGTCGATTCCGT |
| | ATTCCGTGA | AGAGAGA | ATTAGT | CCTGGTGCG | GAATGGGTTGATTAGT |
| | AT (SEQ ID | GATTCTGT | GGTAGA | GG (SEQ ID | GGTAGAAAATGGAAGG |
| | NO: 49) | CCGCGGC | AA (SEQ | NO: 50) | AGAGAGAGATTCTGTC |
| | | (SEQ ID | ID NO: 4) | | CGCGGCCAGGGTTGAT |
| | | NO: 54) | | | TAGTGGTAGAAAAGGG |
| | | | | | TTGATTAGTGGTAGAA |
| | | | | | AGCAGAATTTCCTGGT |
| | | | | | GCGGG (SEQ ID NO: 28) |
| Sgk3 | TGGCCGTCG | GGAGGTG | TGTGGA | GCAGAATTT | TGGCCGTCGATTCCGT |
| | ATTCCGTGA | GAATCTTT | GGGATT | CCTGGTGCG | GAATTGTGGAGGGATT |
| | AT (SEQ ID | TTTTGTAC | GAAGGA | GG (SEQ ID | GAAGGATAATGTGGAG |
| | NO: 49) | GAGGTC | TA (SEQ | NO: 50) | GGATTGAAGGATAAGG |
| | | (SEQ ID | ID NO: 5) | | AGGTGGAATCTTTTTT |
| | | NO: 55) | | | TGTACGAGGTCAATGT |
| | | | | | GGAGGGATTGAAGGAT |
| | | | | | AGCAGAATTTCCTGGT |
| | | | | | GCGGG (SEQ ID NO: 29) |
| Cox5b | TGGCCGTCG | CCCTGTCT | GGGAGA | GCAGAATTT | TGGCCGTCGATTCCGT |
| | ATTCCGTGA | CGAAAAG | ATGAGG | CCTGGTGCG | GAATGGGAGAATGAGG |
| | AT (SEQ ID | CAAACAA | TGTAAT | GG (SEQ ID | TGTAATGTAGGGAGAA |
| | NO: 49) | ATAACAG | GT (SEQ | NO: 50) | TGAGGTGTAATGTACC |
| | | (SEQ ID | ID NO: 6) | | CTGTCTCGAAAAGCAA |
| | | NO: 56) | | | ACAAATAACAGCAGGG |
| | | | | | AGAATGAGGTGTAATG |
| | | | | | TGCAGAATTTCCTGGT |
| | | | | | GCGGG (SEQ ID NO: 30) |
| Gm28376 | TGGCCGTCG | ATGCCAC | TAGAGT | GCAGAATTT | TGGCCGTCGATTCCGT |
| | ATTCCGTGA | TACCACG | TGATAG | CCTGGTGCG | GAATTAGAGTTGATAG |
| | AT (SEQ ID | GAAACCT | AGGGAG | GG (SEQ ID | AGGGAGAAAATGCCAC |
| | NO: 49) | GAGGTTT | AA (SEQ | NO: 50) | TACCACGGAAACCTGA |
| | | T (SEQ ID | ID NO: 7) | | GGTTTTTATAGAGTTG |
| | | NO: 57) | | | ATAGAGGGAGAAATAG |
| | | | | | AGTTGATAGAGGGAGA |
| | | | | | AGCAGAATTTCCTGGT |
| | | | | | GCGGG (SEQ ID NO: 31) |
| Map4k4 | TGGCCGTCG | CTTCACCA | GATGAT | GCAGAATTT | TGGCCGTCGATTCCGT |
| | ATTCCGTGA | GACAGCC | GTAGTA | CCTGGTGCG | GAATGATGATGTAGTA |
| | AT (SEQ ID | TTCTATAA | GTAAGG | GG (SEQ ID | GTAAGGGTACTTCACC |
| | NO: 49) | AGCTGA | GT (SEQ | NO: 50) | AGACAGCCTTCTATAA |
| | | (SEQ ID | ID NO: 8) | | AGCTGAAAGATGATGT |
| | | NO: 58) | | | AGTAGTAAGGGTAGAT |
| | | | | | GATGTAGTAGTAAGGG |
| | | | | | TGCAGAATTTCCTGGT |
| | | | | | GCGGG (SEQ ID NO: 32) |
| 1700019 | TGGCCGTCG | GGTATAG | GGAGTA | GCAGAATTT | TGGCCGTCGATTCCGT |
| D03Rik | ATTCCGTGA | ATGGGCC | GTTGGT | CCTGGTGCG | GAATGGAGTAGTTGGT |
| | AT (SEQ ID | CCATTTCC | TGTTAG | GG (SEQ ID | TGTTAGGAAGGAGTAG |
| | NO: 49) | TCTTACT | GA (SEQ | NO: 50) | TTGGTTGTTAGGAAGG |
| | | (SEQ ID | ID NO: 9) | | TATAGATGGGCCCCAT |
| | | NO: 59) | | | TTCCTCTTACTCAGGA |
| | | | | | GTAGTTGGTTGTTAGG |
| | | | | | AGCAGAATTTCCTGGT |
| | | | | | GCGGG (SEQ ID NO: 33) |
| Zdbf2 | TGGCCGTCG | TCTTGATG | AGGAGG | GCAGAATTT | TGGCCGTCGATTCCGT |
| | ATTCCGTGA | CTGCTGG | AGGGTA | CCTGGTGCG | GAATAGGAGGAGGGTA |
| | AT (SEQ ID | AGTCTCAT | ATGATA | GG (SEQ ID | ATGATAGAATCTTGAT |
| | NO: 49) | CAGCTG | GA (SEQ | NO: 50) | GCTGCTGGAGTCTCAT |
| | | (SEQ ID | ID NO: 10) | | CAGCTGAAAGGAGGAG |
| | | NO: 60) | | | GGTAATGATAGAAAGG |
| | | | | | AGGAGGGTAATGATAG |
| | | | | | AGCAGAATTTCCTGGT |
| | | | | | GCGGG (SEQ ID NO: 34) |
| Inpp4a | TGGCCGTCG | CGTTGTAT | GAGGGT | GCAGAATTT | TGGCCGTCGATTCCGT |
| | ATTCCGTGA | GTGCAGG | TTGTAA | CCTGGTGCG | GAATGAGGGTTTGTAA |
| | AT (SEQ ID | TTTGTGG | GGTGAA | GG (SEQ ID | GGTGAATAAGAGGGTT |
| | NO: 49) | GAACAAA | TA (SEQ | NO: 50) | TGTAAGGTGAATAACG |
| | | (SEQ ID | ID NO: 11) | | TTGTATGTGCAGGTTT |
| | | NO: 61) | | | GTGGGAACAAATAGAG |
| | | | | | GGTTTGTAAGGTGAAT |
| | | | | | AGCAGAATTTCCTGGT |
| | | | | | GCGGG (SEQ ID NO: 35) |
| Gpr45 | TGGCCGTCG | AGGATGA | GAAGTG | GCAGAATTT | TGGCCGTCGATTCCGT |
| | ATTCCGTGA | GGATGGT | AGGTGA | CCTGGTGCG | GAATGAAGTGAGGTGA |
| | AT (SEQ ID | GGTGAAG | TTGAGT | GG (SEQ ID | TTGAGTGAAAGGATGA |
| | NO: 49) | GCCTTGG | GA (SEQ | NO: 50) | GGATGGTGGTGAAGGC |
| | | T (SEQ ID | ID NO: 12) | | CTTGGTTAGAAGTGAG |
| | | NO: 62) | | | GTGATTGAGTGAAGAA |
| | | | | | GTGAGGTGATTGAGTG |
| | | | | | AGCAGAATTTCCTGGT |
| | | | | | GCGGG (SEQ ID NO: 36) |
| Lman21 | TGGCCGTCG | GAGACTA | GGTATT | GCAGAATTT | TGGCCGTCGATTCCGT |
| | ATTCCGTGA | GCCTGTG | ATGTAG | CCTGGTGCG | GAATGGTATTATGTAG |
| | AT (SEQ ID | ACCAATTT | GAAGGT | GG (SEQ ID | GAAGGTGGAGAGACTA |
| | NO: 49) | AACCACA | GG (SEQ | NO: 50) | GCCTGTGACCAATTTA |
| | | (SEQ ID | ID NO: 13) | | ACCACAGAGGTATTAT |
| | | NO: 63) | | | GTAGGAAGGTGGAGGT |
| | | | | | ATTATGTAGGAAGGTG |
| | | | | | GGCAGAATTTCCTGGT |
| | | | | | GCGGG (SEQ ID NO: 37) |
| Ptp4a1 | TGGCCGTCG | GGCGGTT | GAGAAG | GCAGAATTT | TGGCCGTCGATTCCGT |
| | ATTCCGTGA | CATTCGA | TGGTTG | CCTGGTGCG | GAATGAGAAGTGGTTG |
| | AT (SEQ ID | GCCATGT | TAGAGT | GG (SEQ ID | TAGAGTGTAGGCGGTT |
| | NO: 49) | TAATTTAG | GT (SEQ | NO: 50) | CATTCGAGCCATGTTA |
| | | (SEQ ID | ID NO: 14) | | ATTTAGTAGAGAAGTG |
| | | NO: 64) | | | GTTGTAGAGTGTAGAG |
| | | | | | AAGTGGTTGTAGAGTG |
| | | | | | TGCAGAATTTCCTGGT |
| | | | | | GCGGG (SEQ ID NO: 38) |
| Stau2 | TGGCCGTCG | ATCCCTCA | GGTTAG | GCAGAATTT | TGGCCGTCGATTCCGT |
| | ATTCCGTGA | CAGCTCC | TAGGTT | CCTGGTGCG | GAATGGTTAGTAGGTT |
| | AT (SEQ ID | ACAAAAC | GTGGTG | GG (SEQ ID | GTGGTGTTAATCCCTC |
| | NO: 49) | TGCGGGC | TT (SEQ | NO: 50) | ACAGCTCCACAAAACT |
| | | (SEQ ID | ID NO: 15) | | GCGGGCCAGGTTAGTA |
| | | NO: 65) | | | GGTTGTGGTGTTAGGT |
| | | | | | TAGTAGGTTGTGGTGT |
| | | | | | TGCAGAATTTCCTGGT |
| | | | | | GCGGG (SEQ ID NO: 39) |
| Cpa6 | TGGCCGTCG | GAGCCAG | GTATAA | GCAGAATTT | TGGCCGTCGATTCCGT |
| | ATTCCGTGA | CAAACCG | GGTGAT | CCTGGTGCG | GAATGTATAAGGTGAT |
| | AT (SEQ ID | GCAGAAA | TGGTGG | GG (SEQ ID | TGGTGGTGAGAGCCAG |
| | NO: 49) | AGCTGCT | TG (SEQ | NO: 50) | CAAACCGGCAGAAAAG |
| | | G (SEQ ID | ID NO: 16) | | CTGCTGTAGTATAAGG |
| | | NO: 66) | | | TGATTGGTGGTGAGTA |
| | | | | | TAAGGTGATTGGTGGT |
| | | | | | GGCAGAATTTCCTGGT |
| | | | | | GCGGG (SEQ ID NO: 40) |
| Vwc21 | TGGCCGTCG | GGTTTGC | GGAGTA | GCAGAATTT | TGGCCGTCGATTCCGT |
| | ATTCCGTGA | TCATTCTG | GGTTGA | CCTGGTGCG | GAATGGAGTAGGTTGA |
| | AT (SEQ ID | TTGAGCG | TGTGTA | GG (SEQ ID | TGTGTAGTAGGAGTAG |
| | NO: 49) | CGATACA | GT (SEQ | NO: 50) | GTTGATGTGTAGTAGG |
| | | (SEQ ID | ID NO: 17) | | TTTGCTCATTCTGTTG |
| | | NO: 67) | | | AGCGCGATACATAGGA |
| | | | | | GTAGGTTGATGTGTAG |
| | | | | | TGCAGAATTTCCTGGT |
| | | | | | GCGGG (SEQ ID NO: 41) |
| Mcmdc2 | TGGCCGTCG | ATGCAAA | GAGTGT | GCAGAATTT | TGGCCGTCGATTCCGT |
| | ATTCCGTGA | GACATTG | GTGTTA | CCTGGTGCG | GAATGAGTGTGTGTTA |
| | AT (SEQ ID | GCCAGCA | AGGTAG | GG (SEQ ID | AGGTAGGTAGAGTGTG |
| | NO: 49) | TTGCTGT | GT (SEQ | NO: 50) | TGTTAAGGTAGGTAAT |
| | | G (SEQ ID | ID NO: 18) | | GCAAAGACATTGGCCA |
| | | NO: 68) | | | GCATTGCTGTGAAGAG |
| | | | | | TGTGTGTTAAGGTAGG |
| | | | | | TGCAGAATTTCCTGGT |
| | | | | | GCGGG (SEQ ID NO: 42) |
| Tmem14a | TGGCCGTCG | CCCACCC | TGGTTA | GCAGAATTT | TGGCCGTCGATTCCGT |
| | ATTCCGTGA | CCAATAAT | GAGGTT | CCTGGTGCG | GAATTGGTTAGAGGTT |
| | AT (SEQ ID | ACAAATA | AGTGGT | GG (SEQ ID | AGTGGTTGACCCACCC |
| | NO: 49) | AAACCCC | TG (SEQ | NO: 50) | CCAATAATACAAATAA |
| | | (SEQ ID | ID NO: 19) | | AACCCCTATGGTTAGA |
| | | NO: 69) | | | GGTTAGTGGTTGATGG |
| | | | | | TTAGAGGTTAGTGGTT |
| | | | | | GGCAGAATTTCCTGGT |
| | | | | | GCGGG (SEQ ID NO: 43) |
| Sox17 | TGGCCGTCG | GTTCTGCT | ATGTGA | GCAGAATTT | TGGCCGTCGATTCCGT |
| | ATTCCGTGA | GTGCCAA | GTGGTG | CCTGGTGCG | GAATATGTGAGTGGTG |
| | AT (SEQ ID | CCGCTTG | AGAATG | GG (SEQ ID | AGAATGTGAGTTCTGC |
| | NO: 49) | CGTTCGT | TG (SEQ | NO: 50) | TGTGCCAACCGCTTGC |
| | | (SEQ ID | ID NO: 20) | | GTTCGTCAATGTGAGT |
| | | NO: 70) | | | GGTGAGAATGTGAATG |
| | | | | | TGAGTGGTGAGAATGT |
| | | | | | GGCAGAATTTCCTGGT |
| | | | | | GCGGG (SEQ ID NO: 44) |
| Klhdc8a | TGGCCGTCG | CGGGAGA | GGTGGT | GCAGAATTT | TGGCCGTCGATTCCGT |
| | ATTCCGTGA | ACACGGT | TGATTA | CCTGGTGCG | GAATGGTGGTTGATTA |
| | AT (SEQ ID | GGGAGAT | AGGATG | GG (SEQ ID | AGGATGGTACGGGAGA |
| | NO: 49) | GATCTCAT | GT (SEQ | NO: 50) | ACACGGTGGGAGATGA |
| | | (SEQ ID | ID NO: 21) | | TCTCATGAGGTGGTTG |
| | | NO: 71) | | | ATTAAGGATGGTAGGT |
| | | | | | GGTTGATTAAGGATGG |
| | | | | | TGCAGAATTTCCTGGT |
| | | | | | GCGGG (SEQ ID NO: 45) |
| Atp6v1h | TGGCCGTCG | ATGTTAGT | GGAGGT | GCAGAATTT | TGGCCGTCGATTCCGT |
| | ATTCCGTGA | TGGGACA | TAGAAT | CCTGGTGCG | GAATGGAGGTTAGAAT |
| | AT (SEQ ID | GCAGCAT | TTGTGA | GG (SEQ ID | TTGTGAGGAATGTTAG |
| | NO: 49) | CCACAGC | GG (SEQ | NO: 50) | TTGGGACAGCAGCATC |
| | | (SEQ ID | ID NO: 22) | | CACAGCAAGGAGGTTA |
| | | NO: 72) | | | GAATTTGTGAGGAGGA |
| | | | | | GGTTAGAATTTGTGAG |
| | | | | | GGCAGAATTTCCTGGT |
| | | | | | GCGGG (SEQ ID NO: 46) |
| Arid5a | TGGCCGTCG | TTGGTAG | GAGAGA | GCAGAATTT | TGGCCGTCGATTCCGT |
| | ATTCCGTGA | GAGGCAG | GGATTA | CCTGGTGCG | GAATGAGAGAGGATTA |
| | AT (SEQ ID | TGGCTTG | GGTATT | GG (SEQ ID | GGTATTGGAGAGAGAG |
| | NO: 49) | TCGTCCTC | GG (SEQ | NO: 50) | GATTAGGTATTGGATT |
| | | (SEQ ID | ID NO: 23) | | GGTAGGAGGCAGTGG |
| | | NO: 73) | | | CTTGTCGTCCTCCAGA |
| | | | | | GAGAGGATTAGGTATT |
| | | | | | GGGCAGAATTTCCTGG |
| | | | | | TGCGGG (SEQ ID NO: 47) |
| Coq10b | TGGCCGTCG | CCAAGTG | GTAGGT | GCAGAATTT | TGGCCGTCGATTCCGT |
| | ATTCCGTGA | CGGTTTTA | GTTATG | CCTGGTGCG | GAATGTAGGTGTTATG |
| | AT (SEQ ID | CCAAGGT | TTAGGA | GG (SEQ ID | TTAGGAGGACCAAGTG |
| | NO: 49) | TACTATT | GG (SEQ | NO: 50) | CGGTTTTACCAAGGTT |
| | | (SEQ ID | ID NO: 24) | | ACTATTGAGTAGGTGT |
| | | NO: 74) | | | TATGTTAGGAGGAGTA |
| | | | | | GGTGTTATGTTAGGAG |
| | | | | | GGCAGAATTTCCTGGT |
| | | | | | GCGGG (SEQ ID NO: 48) |

TABLE 2
dredFISH labeled binding reagents for brain cell type scanning

| | | Dye and | SEQ IDs of bivalent |
|---|---|---|---|
| Binding target on labeled | | conjugation | binding reagents to |
| binding sequence region; see | Entire labeled binding reagent | chemistry | which it binds (see |
| Table 1) | nucleic acid sequence | used | Table 1) |
| AGAGTGAGTAGTAGTGGAG | ACTCCACTACTACTCACT | Cy5 (via | (SEQ ID NO: 25) |
| T (SEQ ID NO: 1) | CT (SEQ ID NO: 75) | Disulfide) | |
| TGTGATGGAAGTTAGAGGG | ACCCTCTAACTTCCATCA | Cy5 (via | (SEQ ID NO: 26) |
| T (SEQ ID NO: 2) | CA (SEQ ID NO: 76) | Disulfide) | |
| TGAAAGGAATGGGTTGTGG | ACCACAACCCATTCCTTT | Cy5 (via | (SEQ ID NO: 27) |
| T (SEQ ID NO: 3) | CA (SEQ ID NO: 77) | Disulfide) | |
| GGGTTGATTAGTGGTAGAA | TTTCTACCACTAATCAAC | Cy5 (via | (SEQ ID NO: 28) |
| A (SEQ ID NO: 4) | CC (SEQ ID NO: 78) | Disulfide) | |
| TGTGGAGGGATTGAAGGAT | TATCCTTCAATCCCTCCA | Cy5 (via | (SEQ ID NO: 29) |
| A (SEQ ID NO: 5) | CA (SEQ ID NO: 79) | Disulfide) | |
| GGGAGAATGAGGTGTAATG | ACATTACACCTCATTCTC | Cy5 (via | (SEQ ID NO: 30) |
| T (SEQ ID NO: 6) | CC (SEQ ID NO: 80) | Disulfide) | |
| TGGTTAGAGGTTAGTGGTT | CAACCACTAACCTCTAAC | Cy5 (via | (SEQ ID NO: 31) |
| G (SEQ ID NO: 7) | CA (SEQ ID NO: 81) | Disulfide) | |
| ATGTGAGTGGTGAGAATGT | CACATTCTCACCACTCAC | Cy5 (via | (SEQ ID NO: 32) |
| G (SEQ ID NO: 8) | AT (SEQ ID NO: 82) | Disulfide) | |
| GGTGGTTGATTAAGGATGG | ACCATCCTTAATCAACCA | Cy5 (via | (SEQ ID NO: 33) |
| T (SEQ ID NO: 9) | CC (SEQ ID NO: 83) | Disulfide) | |
| TAGAGTTGATAGAGGGAGA | TTCTCCCTCTATCAACTCT | Cy5 (via | (SEQ ID NO: 34) |
| A (SEQ ID NO: 10) | A (SEQ ID NO: 84) | Disulfide) | |
| GATGATGTAGTAGTAAGGG | ACCCTTACTACTACATCA | Cy5 (via | (SEQ ID NO: 35) |
| T (SEQ ID NO: 11) | TC (SEQ ID NO: 85) | Disulfide) | |
| GGAGTAGTTGGTTGTTAGG | TCCTAACAACCAACTACT | Cy5 (via | (SEQ ID NO: 36) |
| A (SEQ ID NO: (12) | CC (SEQ ID NO: 86) | Disulfide) | |
| AGGAGGAGGGTAATGATAG | TCTATCATTACCCTCCTC | Cy5 (via | (SEQ ID NO: 37) |
| A (SEQ ID NO: 13) | CT (SEQ ID NO: 87) | Disulfide) | |
| GAGGGTTTGTAAGGTGAAT | TATTCACCTTACAAACCC | Cy5 (via | (SEQ ID NO: 38) |
| A (SEQ ID NO: 14) | TC (SEQ ID NO: 88) | Disulfide) | |
| GAAGTGAGGTGATTGAGTG | TCACTCAATCACCTCACT | Cy5 (via | (SEQ ID NO: 39) |
| A (SEQ ID NO: 15) | TC (SEQ ID NO: 89) | Disulfide) | |
| GGAGGTTAGAATTTGTGAG | CCTCACAAATTCTAACCT | Cy5 (via | (SEQ ID NO: 40) |
| G (SEQ ID NO: 16) | CC (SEQ ID NO: 90) | Disulfide) | |
| GAGAGAGGATTAGGTATTG | CCAATACCTAATCCTCTC | Cy5 (via | (SEQ ID NO: 41) |
| G (SEQ ID NO: 17) | TC (SEQ ID NO: 91) | Disulfide) | |
| GTAGGTGTTATGTTAGGAG | CCTCCTAACATAACACCT | Cy5 (via | (SEQ ID NO: 42) |
| G (SEQ ID NO: 18) | AC (SEQ ID NO: 92) | Disulfide) | |
| GGTATTATGTAGGAAGGTG | CCACCTTCCTACATAATA | Cy5 (via | (SEQ ID NO: 43) |
| G (SEQ ID NO: 19) | CC (SEQ ID NO: 93) | Disulfide) | |
| GAGAAGTGGTTGTAGAGTG | ACACTCTACAACCACTTC | Cy5 (via | (SEQ ID NO: 44) |
| T (SEQ ID NO: 20) | TC (SEQ ID NO: 94) | Disulfide) | |
| GGTTAGTAGGTTGTGGTGT | AACACCACAACCTACTAA | Cy5 (via | (SEQ ID NO: 45) |
| T (SEQ ID NO: 21) | CC (SEQ ID NO: 95) | Disulfide) | |
| GTATAAGGTGATTGGTGGT | CACCACCAATCACCTTAT | Cy5 (via | (SEQ ID NO: 46) |
| G (SEQ ID NO: 22) | AC (SEQ ID NO: 96) | Disulfide) | |
| GGAGTAGGTTGATGTGTAG | ACTACACATCAACCTACT | Cy5 (via | (SEQ ID NO: 47) |
| T (SEQ ID NO: 23) | CC (SEQ ID NO: 97) | Disulfide) | |
| GAGTGTGTGTTAAGGTAGG | ACCTACCTTAACACACAC | Cy5 (via | (SEQ ID NO: 48) |
| T (SEQ ID NO: 24) | TC (SEQ ID NO: 98) | Disulfide) | |

The following oligonucleotides were prepared following the same methods described above, and conjugated to Cy5 dye. The 24 labeled binding reagents were used to detect the more than 92,000 bivalent binding reagents used in the examples described herein. Some examples of the bivalent binding reagents, their target molecular markers, molecular marker binding region sequence and labeled-binding-reagent binding region sequences are set forth in Table 1.

What is claimed is:

1. A method for identifying the specific locations of a plurality of specific cell types within a population of cells in a biological specimen, the method comprising the steps of:
a. selecting the plurality of specific cell types within the specimen, based on the origin of the specimen and the known cell types anticipated to be present therein;
b. determining among those specific cell types the known extent of presence or lack thereof of one or more known molecular markers of each specific cell type therein;
c. establishing a relationship using a subset of the one or more molecular markers among all specific cell types therein, wherein a weight given to the extent of expression or lack thereof of each molecular marker by each of the specific cell types using a linear dimensionality reduction process differentiates each specific cell type from each other specific cell type;
d. preparing a set of bivalent binding reagents and a set of labeled binding reagents, wherein each bivalent binding reagent is specific to a molecular marker and comprises a molecular marker-binding region and at least one labeled-binding-reagent binding region, wherein one or more of each molecular marker-specific bivalent binding reagent is provided to bind to a specific molecular marker to an extent to differentiate the specific molecular marker from each other molecular marker, and wherein each labeled binding reagent is detectably labeled by individually and/or simultaneously detectable labels, such that the set of bivalent binding reagents and labeled binding reagents bound thereto, when bound to the subset of molecular markers expressed by each specific cell type in the sample, provides an extent of labeling that differentiates each specific cell type from each other specific cell type;
e. staining the specimen with the bivalent binding reagents and the labeled binding reagents;
f. imaging positions throughout the specimen to detect the labeled binding reagents and extent of labeling thereof sequentially or simultaneously, to provide the positions of the subset of molecular markers and extent of expression thereof;
g. correlating the extent of expression of the subset of molecular markers at positions throughout the specimen using the linear dimensionality reduction process based on the established relationship between the extent of expression of each of the plurality of molecular markers or lack thereof by each of the specific cell types that differentiates each specific cell type from each other specific cell type, to establish a highest-probability estimate of the presence of a specific cell type at a specific location within the specimen; and
h. identifying the specific locations of the specific estimated cell types within the specimen.

2. The method of claim 1 wherein the known molecular markers are nucleic acid polymers.

3. The method of claim 2 wherein the nucleic acid polymers comprise RNA.

4. The method of claim 1 wherein the known molecular markers are peptides, whole proteins, and/or protein fragments.

5. The method of claim 4 wherein the proteins are any of a peptide, nuclear protein, cytosolic protein, mitochondrial protein, secreted protein, cell-surface protein, receptor, transcription factor, antibody, or any combination thereof.

6. The method of claim 1 wherein the molecular markers are selected from among metabolites, lipids, carbohydrates including polysaccharides, glycolipids, vitamins, fatty acids, co-factors, pigments, metals, or any other biochemicals or compounds, organic or inorganic, found within a biological system.

7. The method of claim 1 wherein step (b) is accomplished using scRNAseq.

8. The method of claim 1 wherein steps (a), (b) and (c) are carried out in silico.

9. The method of claim 1 the dimensionality reduction process is principal component analysis (PCA), discernment projection non-negative matrix factorization (dPNMF) or an artificial neural network based design.

10. The method of claim 1 wherein step (b) further includes organizing the specific cell types into a hierarchical taxonomy according to the plurality of known molecular markers.

11. The method of claim 1 wherein step (c) is accomplished using recursive partitioning.

12. The method of claim 10 wherein the number of detectable labels of step (d) is equal to the number of hierarchical levels of the hierarchical taxonomy of step (b).

13. The method of claim 9 wherein the dimensionality reduction process uses recursive partitioning.

14. The method of claim 9 wherein dPNMF comprises
a. fitting a dPNMF model to training data;
b. fitting a classifier to one class per cell type; and
c. creating a staining profile for each cell type according to a weighting, whereby the number of cell labels per molecular marker approximates weighting.

15. The method of claim 14 wherein the classifier is a Naïve Bayesian classifier.

16. The method of claim 1 wherein multiple bivalent binding reagents are provided that bind to the same molecular marker.

17. The method of claim 16, wherein the multiple bivalent reagents that bind to the same molecular marker have the same one or more, or different, label-binding-reagent-binding regions.

18. The method of claim 1 wherein the labeled binding reagent comprises a label and a oligonucleotide that binds to an oligonucleotide bivalent binding reagent.

19. The method of claim 1 wherein one or more bivalent binding reagents comprise a molecular-marker binding region and at least one labeled-binding-reagent binding region.

20. The method of claim 19 wherein one or more bivalent binding reagents comprise at least one molecular-marker binding region nucleic acid sequence, and at least one labeled-binding-reagent binding region nucleic acid sequence.

21. The method of claim 19 wherein the bivalent binding reagent comprises two labeled-binding-reagent binding sequences, binding the same or different labeled binding reagents.

22. The method of claim 19 wherein the bivalent binding reagent comprises three labeled-binding-reagent binding sequences.

23. The method of any one of claims 19-22 wherein the labeled-binding-reagent binding sequences bind one or more of the same, or two different, or two of the same and one different, or three all different labeled binding reagents, or any other combination thereof.

24. The method of claim 19 wherein the bivalent binding reagent comprises amplification sequences.

25. The method of claim 24 wherein the amplification sequence are removed before incubating.

26. The method of claim 23 wherein a forward amplification sequence is provided at the 5′ end of the bivalent binding reagent, and a reverse amplification sequence at the 3′end.

27. The method of claim 23 wherein bivalent binding sequence comprises, from 5′ to 3′, a forward amplification sequence, a molecular-marker binding sequence, a labeled-binding-reagent binding sequence, and a reverse amplification sequence.

28. The method of claim 23 wherein the bivalent binding sequence comprises, from 5′ to 3′, a forward amplification sequence, a labeled-binding-reagent binding sequence, a molecular-marker binding sequence, and a reverse amplification sequence.

29. The method of claim 23 wherein the bivalent binding sequence comprises from 5′ to 3′, a forward amplification sequence, a labeled-binding-reagent binding sequence, a same or different labeled-binding-reagent binding sequence, a molecular-marker binding sequence, and a reverse amplification sequence.

30. The method of claim 23 wherein the bivalent binding sequence comprises, from 5′ to 3′, a forward amplification sequence, a labeled-binding-reagent binding sequence, a same or different labeled-binding-reagent binding sequence, a molecular-marker binding sequence, a same or different labeled-binding-reagent binding region, and a reverse amplification sequence.

31. The method of claim 23 wherein the bivalent binding sequence comprises, from 5′ to 3′, a forward amplification sequence, a labeled-binding-reagent binding sequence, a molecular-marker binding sequence, a same or different labeled-binding-reagent binding region, a same or different labeled-binding-reagent binding sequence, and a reverse amplification sequence.

32. The method of any one of claims 23-31 wherein one or more additional nucleotides (e.g., A, C, G, and/or T) may be provided as spacers between any of the aforementioned regions, such as one or more A.

33. The method of claim 18 wherein the labeled binding reagents comprises an oligonucleotide sequence that binds to a labeled-binding-reagent binding sequence of a bivalent binding reagent, and a detectable label, such as a fluorescent dye.

34. The method of claim 33 wherein the dye is covalent bound to the oligonucleotide region of the labeled binding reagent.

35. The method of claim 33, wherein the dye is reversibly bound to the oligonucleotide region of the labeled binding reagent.

36. The method of claim 35 where the reversibly bound dye comprises a disulfide bond.

37. The method of claim 1 wherein the labeled binding reagent comprises a due selected from among Cy5, BODIPY 630/650-X, LC Red 640, Alexa Fluor 633, BODIPY 650/665-X, Alexa Fluor 647, Alexa Fluor 660, Cyanine5.5, Alexa Fluor 680, Alexa Fluor 700 and Alexa Fluor 750.

38. The method of claim 1 wherein step (f) is accomplished by hyperspectral scanning standard fluorescence imaging, light sheet imaging, or flow cytometry, mass spectrometry, or optical sensing.

39. The method of claim 1 wherein the imaging at positions throughout the specimen to detect each labeled binding reagent and extent of labeling is obtained sequentially

40. The method of claim 1 wherein the imaging at positions throughout the specimen to detect the labeled binding reagents and extent of labeling is obtained simultaneously.

41. The method of claim 1, wherein the specimen is incubated with all of the bivalent binding reagents and then sequentially incubated with one or a subset of the labeled binding reagents.

42. The method of claim 1, wherein the specimen is incubated with all of the bivalent binding reagents and all of the labeled binding reagents simultaneously.

43. The method of claim 41, wherein the specimen is imaged after each sequential incubation with each labeled binding reagent or subset of the labeled binding reagents.

44. The method of claim 1 wherein the imaging is performed batchwise to detect one or more dyes during each sequential imaging.

45. The method of claim 41 wherein after each imaging of the sample, the one or more labeled binding reagents are washed out of the specimen before the next one or more labeled binding reagents are incubated then imaged.

46. The method of claim 45, wherein the washing out comprises removing the labeled binding reagents.

47. The method of claim 46 wherein the washing out comprises reducing a disulfide that is binding the dye to the labeled binding reagent, and washing the specimen.

48. The method of claim 1 wherein the imaging is low magnification imaging.

49. The method of claim 1 wherein the specimen is cleared before incubation or imaging.

50. The method of claim 1 wherein the specimen is embedded in a hydrogel before incubating or imaging.

51. The method of claim 1 wherein the specimen is brain.

52. The method of claim 1 wherein the set of bivalent binding reagents comprises one or more of SEQ ID NOs:25-48.

53. The method of claim 1 wherein the set of labeled binding reagents comprises one or more of SEQ ID NOs:75-98.

54. The method of claim 51 wherein the set of bivalent binding reagents comprise one or more of SEQ ID NOs:25-48 and the set of labeled binding reagents comprise one or more of SEQ ID NOs:75-98 that bind to the one or more of SEQ ID NOs:25-48.

55. The method of claim 1 where the specimen is a whole organ.

56. The method of claim 1 wherein the specimen is fresh, frozen, formalin preserved, alcohol preserved, a thin section, a thick section, a biopsy specimen or a previously formalin-fixed, paraffin embedded specimen.

57. The method of claim 1 wherein the specimen is obtained from a patient, a healthy subject, a pathology specimen, a fossilized specimen, a frozen or cryogenically preserved specimen, an exhumed specimen or a mummified specimen.

58. The method of claim 1 wherein the bivalent binding reagent comprises an antibody or antigen-binding fragment.

59. A bivalent binding reagent selected from among SEQ ID NOs:25-48.

60. A labeled binding reagent selected from among SEQ ID NOs:75-98.
